(19) United States
(12) Patent Application Publication
(10) Pub. No.: US 2024/0041439 A1
(43) Pub. Date: Feb. 8, 2024

(54) SYSTEMS AND METHODS FOR PRESERVATION OF A CERVICOVAGINAL FLUID SAMPLE

(71) Applicant: NextGen Jane, Inc., Oakland, CA (US)

(72) Inventors: Ridhi TARIYAL, Oakland, CA (US); Stephen GIRE, Oakland, CA (US); Adrienne CLARK, Oakland, CA (US); Sarah BUSH, Oakland, CA (US); Remi Ana GODINEZ, Oakland, CA (US)

(21) Appl. No.: 18/258,714

(22) PCT Filed: Dec. 21, 2021
(86) PCT No.: PCT/US2021/064692

§ 371 Date: Jun. 21, 2023

Related U.S. Application Data
(60) Provisional application No. 63/130,240, filed on Dec. 23, 2020.

(51) Int. Cl. A61B 10/00; B01L 3/00

(52) U.S. Cl. CPC A61B 10/0045; B01L 3/5029; B01L 2300/042; B01L 2300/0672; B01L 2400/0677; B01L 2200/16; A61B 2010/0074

(57) ABSTRACT

Provided are systems and methods for preserving a cervico-vaginal fluid sample.

### CROSS REFERENCE

This application claims the benefit of U.S. Provisional Application No. 63/130,240 filed Dec. 23, 2020, which is incorporated herein by reference in its entirety.

### SUMMARY

In certain aspects, disclosed herein is a system for extracting and preserving components of a biological sample of a subject comprising: a sample collector that collects and retains the biological sample from a vaginal canal of the subject; and a sample receptacle; wherein the sample receptacle comprises a first chamber that is configured to hold the sample collector; a second chamber that is configured to contain a stabilization buffer; a disruptable member between the first chamber and the second chamber; and a displaceable shuttle, wherein displacement of the displaceable shuttle from the first chamber towards the second chamber is configured to disrupt the disruptable member such that there is fluid communication between the first chamber and the second chamber.

In some embodiments, the sample collector comprises an absorbent-diffuse material that collects, retains or releases the biological material. In some embodiments, the absorbent-diffuse material comprises one or more of a plant fiber material, a disposable material, a flushable material, a biodegradable material, and an organic material. In some embodiments, the sample collector is insertable in the vaginal canal. In some embodiments, the sample collector is a tampon, a pad, a plug, or a swab. In some embodiments, the sample collector continuously interacts with the stabilization buffer after the disruptable member is disrupted. In some embodiments, the sample collector is in continuous communication with the stabilization buffer for about 10 minutes to about 48 hours. In some embodiments, the sample collector is not compressed. In some embodiments, the displaceable shuttle is configured such that it does not compress the sample collector. In some embodiments, the biological sample is released from the sample collector into the stabilization buffer. In some embodiments, the system further comprises a locking ring comprising a first detent and a second detent, wherein the locking ring is located within the first chamber. In some embodiments, the displaceable shuttle comprises a first end and a second end; wherein the first end of the displaceable shuttle comprises ridges; wherein the displaceable shuttle is retained in the first chamber by the ridges fitting into the first detent on the locking ring; wherein the second end of the displaceable shuttle comprises a piercing end configured to disrupt the disruptable member. In some embodiments, the displaceable shuttle is configured to hold the sample collector within the first chamber. In some embodiments, placing a cap on the receptacle displaces the displaceable shuttle from the first detent on the locking ring to the second detent on the locking ring. In some embodiments, the cap is configured to engage the shuttle before fully closing. In some embodiments, the cap comprises a material that is softer than the receptacle. In some embodiments, the ridges of the displaceable shuttle are configured to prevent the displaceable shuttle from piercing the disruptable member before the cap is placed. In some embodiments, the second chamber comprises a buffer cup configured to contain the stabilization buffer. In some embodiments, the buffer cup comprises an outer ring and an inner ring, wherein the outer ring is larger in diameter than the inner ring. In some embodiments, the buffer cup is configured to contain about 10 milliliters to 30 milliliters of the stabilization buffer. In some embodiments, the disruptable member is attached to the buffer cup. In some embodiments, the disruptable member comprises a foil lid or a plastic film. In some embodiments, the disruptable member is attached to the buffer cup by a method selected from the group consisting of heat sealing, adhesion, curing, and welding. In some embodiments, the sample receptacle comprises a compression mechanism configured to compress the sample collector. In some embodiments, the sample receptacle does not comprise a compression mechanism configured to compress the sample collector.

In some embodiments, the system further comprises a harvester mechanism. In some embodiments, the harvester mechanism comprises a cap and a stem, wherein the stem is configured to be inserted into the sample receptacle and to compress the sample collector. In some embodiments, the harvester mechanism comprises snap arms that interface with the sample collector. In some embodiments, the snap arms of the harvester are configured to engage a lip on the sample receptacle to retain the sample collector within the displaceable shuttle. In some embodiments, the harvester is pressed onto the sample receptacle using an arbor press. In some embodiments, the harvester mechanism comprises a fluid passageway configured to allow the stabilization buffer and the biological sample to be moved from the second chamber of the sample receptacle into a second receptacle. In some embodiments, the harvester comprises an air passageway that is separate from the fluid passageway. In some embodiments, the fluid passageway is selected from the group consisting of a spout, a luer lock, a syringe port, and a one-way valve.

In some embodiments, the stabilization buffer comprises a preservative. In some embodiments, the stabilization buffer is formulated to preserve one or more of intact cells, nucleic acids, DNA, RNA, protein or metabolite(s). In some embodiments, the system is configured to preserve one or more of more of intact cells, nucleic acids, DNA, RNA, protein or metabolite(s) for at least 72 hours at room temperature. In some embodiments, upon disruption of the disruptable member, the system is configured to retain the stabilization buffer between the first chamber and the second chamber when the device is rotated, placed horizontally, and/or inverted from its vertical position. In some embodiments, upon rotation, horizontal placement, or inversion, the device is configured such that the stabilization buffer washes over the sample collector thereby releasing the biological sample from the sample collector into the stabilization buffer. In some embodiments, the displaceable shuttle comprises a first notch or a first plurality of notches. In some embodiments, the cap of the sample receptacle comprises a second notch or second plurality of notches. In some embodiments, the first notch or plurality of notches is configured to catch a string appended to one end of the sample collector when the cap is placed on sample receptacle. In some embodiments, the second notch or plurality of notches is configured to hold a string appended to one end of the sample collector when the cap is screwed on to the sample receptacle. In some embodiments, after placing a cap on the sample receptacle, the string appended to one end of the sample collector remains entirely within the sample receptacle.

In some embodiments, disclosed herein is a kit comprising: the system described herein and instructions for the use of the kit. In some embodiments, the kit further comprises a biohazard bag and an absorbent material. In some embodiments, the kit further comprises gloves. In some embodiments, the contents are sterile and free of debris. In some embodiments, the kit comprises a retainer or snap-fit lid, wherein the retainer or snap-fit lid seals the system from foreign materials.

In some embodiments, disclosed herein is a sample receptacle comprising: a tampon, wherein the tampon comprises a string; a displaceable shuttle, wherein the displaceable shuttle comprises a first notch or a first plurality of notches, wherein the first notch or first plurality of notches is configured to catch the tampon string; and a cap, wherein the cap comprises a second notch or a second plurality of notches, wherein said second notch or second plurality of notches is configured to hold the tampon string when the cap is screwed on to the sample receptacle.

In some embodiments, the tampon string is fully contained within the sample receptacle after the cap is engaged with the sample receptacle. In some embodiments, the first notch or first plurality of notches is V-shaped. In some embodiments, the second notch or second plurality of notches is V-shaped.

In certain aspects, disclosed herein is a system for extracting and preserving components of a biological sample of a subject comprising: a sample collector that collects and retains the biological sample from a vaginal canal of the subject; and a sample receptacle; wherein the sample receptacle comprises a first chamber that is configured to hold the sample collector; a second chamber that is configured to contain a stabilization buffer; a disruptable member between the first chamber and the second chamber; and a piercer, wherein the piercer is configured to pierce the disruptable member upon contact with said second chamber such that there is fluid communication between the first chamber and the second chamber.

In some embodiments, the sample collector comprises an absorbent-diffuse material that collects, retains or releases the biological material. In some embodiments, the absorbent-diffuse material comprises one or more of a plant fiber material, a disposable material, a flushable material, a biodegradable material, and an organic material. In some embodiments, the sample collector is insertable in the vaginal canal. In some embodiments, the sample collector is a tampon, a pad, a plug, or a swab. In some embodiments, the sample collector continuously interacts with the stabilization buffer after the disruptable member is disrupted. In some embodiments, the sample collector is in continuous communication with the stabilization buffer for about 10 minutes to about 48 hours. In some embodiments, the sample collector is not compressed. In some embodiments, the piercer is configured such that it does not compress the sample collector. In some embodiments, the biological sample is released from the sample collector into the stabilization buffer. In some embodiments, the piercer comprises a piercing end configured to disrupt the disruptable member. In some embodiments, the piercer is configured to hold the sample collector within the first chamber. In some embodiments, the second chamber is located within a cap of said sample receptacle. In some embodiments, the cap comprises a buffer cup configured to contain the stabilization buffer. In some embodiments, the buffer cup comprises an outer ring and an inner ring, wherein the outer ring is larger in diameter than the inner ring. In some embodiments, the buffer cup is configured to contain about 10 milliliters to 30 milliliters of the stabilization buffer. In some embodiments, the disruptable member is attached to the buffer cup. In some embodiments, the disruptable member comprises a foil lid or a plastic film. In some embodiments, the disruptable member is attached to the buffer cup by a method selected from the group consisting of heat sealing, adhesion, curing, and welding. In some embodiments, the sample receptacle comprises a compression mechanism configured to compress the sample collector. In some embodiments, the sample receptacle does not comprise a compression mechanism configured to compress the sample collector.

In some embodiments, the system further comprises a harvester mechanism. In some embodiments, the harvester mechanism comprises a cap and a stem, wherein the stem is configured to be inserted into the sample receptacle and to compress the sample collector. In some embodiments, the harvester mechanism comprises snap arms that interface with the sample collector. In some embodiments, the snap arms of the harvester are configured to engage a lip on the sample receptacle to retain the sample collector within the piercer. In some embodiments, the harvester is pressed onto the sample receptacle using an arbor press. In some embodiments, the harvester mechanism comprises a fluid passageway configured to allow the stabilization buffer and the biological sample to be moved from the second chamber of the sample receptacle into a second receptacle. In some embodiments, the harvester comprises an air passageway that is separate from the fluid passageway. In some embodiments, the fluid passageway is selected from the group consisting of a spout, a luer lock, a syringe port, and a one-way valve.

In some embodiments, the stabilization buffer comprises a preservative. In some embodiments, the stabilization buffer is formulated to preserve one or more of intact cells, nucleic acids, DNA, RNA, protein or metabolite(s). In some embodiments, the system is configured to preserve one or more of more of intact cells, nucleic acids, DNA, RNA, protein or metabolite(s) for at least 72 hours at room temperature. In some embodiments, upon disruption of the disruptable member, the system is configured to retain the stabilization buffer between the first chamber and the second chamber when the device is rotated, placed horizontally, and/or inverted from its vertical position. In some embodiments, upon rotation, horizontal placement, or inversion, the device is configured such that the stabilization buffer washes over the sample collector thereby releasing the biological sample from the sample collector into the stabilization buffer. In some embodiments, the piercer comprises a first notch or a first plurality of notches. In some embodiments, the cap of the sample receptacle comprises a second notch or second plurality of notches. In some embodiments, the first notch or plurality of notches is configured to catch a string appended to one end of the sample collector when the cap is placed on sample receptacle. In some embodiments, the second notch or plurality of notches is configured to hold a string appended to one end of the sample collector when the cap is screwed on to the sample receptacle. In some embodiments, after placing a cap on the sample receptacle, the string appended to one end of the sample collector remains entirely within the sample receptacle.

In some embodiments, disclosed herein is a kit comprising: the system described herein and instructions for the use of the kit. In some embodiments, the kit further comprises a biohazard bag and an absorbent material. In some embodiments, the kit further comprises gloves. In some embodiments, the contents are sterile and free of debris. In some embodiments, the kit comprises a retainer or snap-fit lid, wherein the retainer or snap-fit lid seals the system from foreign materials.

In some embodiments, disclosed herein is a sample receptacle comprising: a tampon, wherein the tampon comprises a string; a piercer, wherein the piercer comprises a first notch or a first plurality of notches, wherein the first notch or first plurality of notches is configured to catch the tampon string; and a cap, wherein the cap comprises a second notch or a second plurality of notches, wherein said second notch or second plurality of notches is configured to hold the tampon string when the cap is screwed on to the sample receptacle.

In some embodiments, the tampon string is fully contained within the sample receptacle after the cap is engaged with the sample receptacle. In some embodiments, the first notch or first plurality of notches is V-shaped. In some embodiments, the second notch or second plurality of notches is V-shaped.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1D depict a sample receptacle comprising a buffer cup, a locking ring, and a displaceable shuttle. FIG. 1A depicts a top view; FIG. 1B depicts a cross section; FIG. 1C depicts an exploded view of a sample receptacle comprising a buffer cup, a locking ring, and a displaceable shuttle; and FIG. 1D depicts a detailed inset.

FIG. 2A depicts a sample receptacle, cap, and sample collector prior to engaging the cap. FIG. 2B depicts a sample receptacle, cap, and sample collector after engaging the cap.

FIGS. 3A-3D depict a sample receptacle. FIG. 3A depicts a top view of a sample receptacle; FIG. 3B depicts a cross section of a sample receptacle; FIG. 3C depicts a detailed inset of FIG. 3B; and FIG. 3D depicts a three-dimensional rendering of a sample receptacle.

FIGS. 4A-4C depict a cap. FIG. 4A depicts a top view of a cap; FIG. 4B depicts a cross section of a cap; and FIG. 4C depicts a three-dimensional rendering of a cap.

FIGS. 5A-5D depict a locking ring. FIG. 5A depicts a top view of a locking ring; FIG. 5B depicts a front view of a locking ring; FIG. 5C depicts a cross section of a locking ring; and FIG. 5D depicts a three-dimensional rendering of a locking ring.

FIGS. 6A-6D depict a displaceable shuttle. FIG. 6A depicts a top view of a displaceable shuttle; FIG. 6B depicts a cross section of a displaceable shuttle; FIG. 6C depicts a detailed inset of a displaceable shuttle; and FIG. 6D depicts a three-dimensional rendering of a displaceable shuttle.

FIGS. 7A-7F depict a buffer cup and disruptable member. FIG. 7A depicts a top view of a buffer cup; FIG. 7B depicts a detailed inset of FIG. 7A; FIG. 7C depicts a cross section of a buffer cup and a disruptable member; FIG. 7D depicts a side view and a top view of a disruptable member; FIG. 7E depicts a side view of a buffer cup and a disruptable member; and FIG. 7F depicts a three dimensional rendering of a buffer cup and a disruptable member.

FIGS. 8A-8C depict a harvester mechanism. FIG. 8A depicts a top view of a harvester mechanisms; FIG. 8B depicts a cross section of a harvester mechanism; and FIG. 8C depicts a detailed inset and a side view of a harvester mechanism.

FIGS. 9A-9D depict the kit described herein. FIG. 9A is a photograph of the assembled kit. FIG. 9B depicts removing the retainer holding the sample receptacle, cap, and gloves in place. FIG. 9C depicts sealing the sample collector containing the sample and the sample collector in a biohazard bag; FIG. 9D depicts placing the biohazard bag within the kit for mailing to the processing facility.

FIG. 10A depicts a cap containing notches, a displaceable shuttle containing notches, a buffer cup, and a sample receptacle; FIG. 10B depicts a sample receptacle comprising a displaceable shuttle containing notches, a buffer cup, and a cap containing notches prior to engagement of the cap with the sample receptacle; FIG. 10C depicts a sample receptacle comprising a displaceable shuttle containing notches, a buffer cup, a cap containing notches, and a sample collector prior to engagement of the cap with the sample receptacle; FIG. 10D depicts a sample receptacle comprising a displaceable shuttle containing notches, a buffer cup, a cap containing notches, and a sample collector after engagement of the cap with the sample receptacle.

FIG. 11A depicts a cap containing notches, a piercer containing notches, and a sample receptacle; FIG. 11B depicts a sample receptacle comprising a piercer containing notches and a cap containing notches prior to the engagement of the cap with the sample receptacle; FIG. 11C depicts a sample receptacle comprising a piercer containing notches, a cap containing notches, and a sample collector prior to engagement of the cap with the sample receptacle; FIG. 11D depicts a sample receptacle comprising a piercer containing notches, a cap containing notches, and a sample collector after engagement of the cap with the sample receptacle.

FIG. 12A depicts a sample receptacle, a cap containing notches, and a tampon with a string attached prior to engaging the cap. FIG. 12B depicts a sample receptacle, a cap containing notches, and a tampon with a string attached with the cap placed on the sample receptacle. FIG. 12C depicts a sample receptacle, a cap containing notches, and a tampon with a string attached while the cap begins to be screwed onto the sample receptacle. FIG. 12D depicts a sample receptacle, a cap containing notches, and a tampon with a string attached while the cap is screwed onto the sample receptacle. FIG. 12E depicts a sample receptacle, a cap containing notches, and a tampon with a string attached while the cap continues to be screwed onto the sample receptacle. FIG. 12F depicts a sample receptacle, a cap containing notches, and a tampon with a string attached while the cap continues to be screwed onto the sample receptacle. FIG. 12G depicts a sample receptacle, a cap containing notches, and a tampon with a string attached after the cap is fully engaged.

FIGS. 13A-13D depict a harvester mechanism. FIG. 13A depicts two harvester components—the cap and the stem. FIG. 13B depicts a cross section of a harvester mechanism. FIG. 13C depicts the harvester mechanism and a sample receptacle prior to the engagement of the harvester with the sample receptacle. FIG. 13D depicts the harvester mechanism that is fully engaged with the sample receptacle.

### DETAILED DESCRIPTION

Provided herein are systems and methods for preservation of a cervicovaginal fluid sample.

### I. System for Collection of a Menstrual Sample

A. Sample Receptacle

In some embodiments, systems and methods described herein comprise a sample receptacle. In some embodiments, the sample receptacle comprises a first chamber that is configured to hold the sample collector; a second chamber that is configured to contain a stabilization buffer; a disruptable member between the first chamber and the second chamber; and a displaceable shuttle, wherein displacement of the displaceable shuttle from the first chamber to the second chamber is configured to disrupt the disruptable member such that there is fluid communication between the first chamber and the second chamber. In some embodiments, the sample receptacle comprises an outer bottle; a buffer cup comprising a stabilization buffer, wherein the buffer cup is sealed with a disruptable member; and a displaceable shuttle configured to hold the sample collector, wherein the displaceable shuttle is configured to disrupt the disruptable member such that there is fluid communication between the sample collector and the buffer cup.

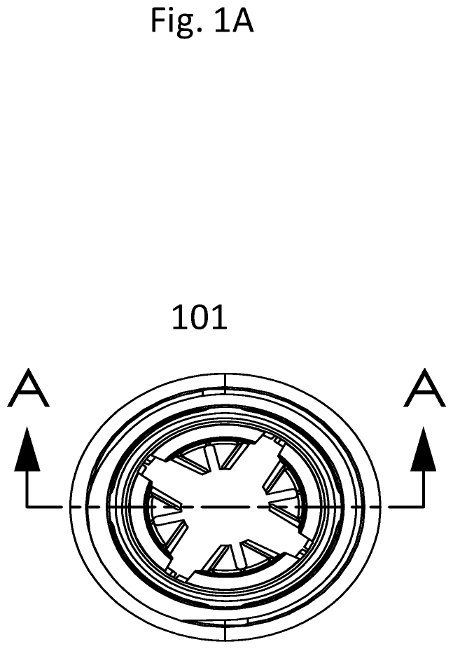

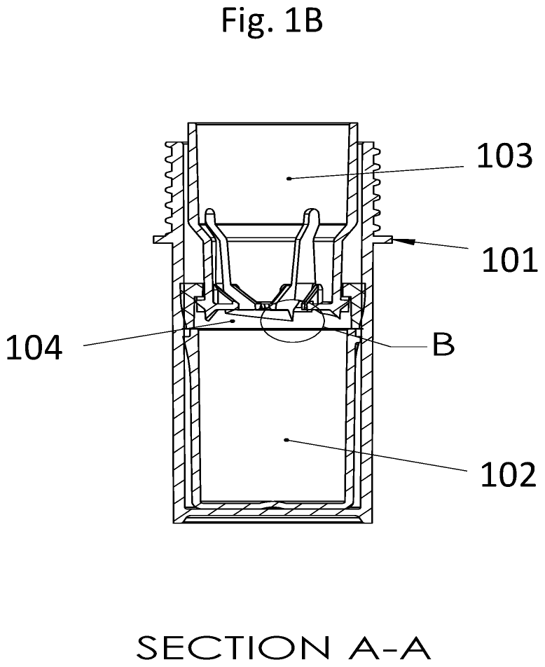

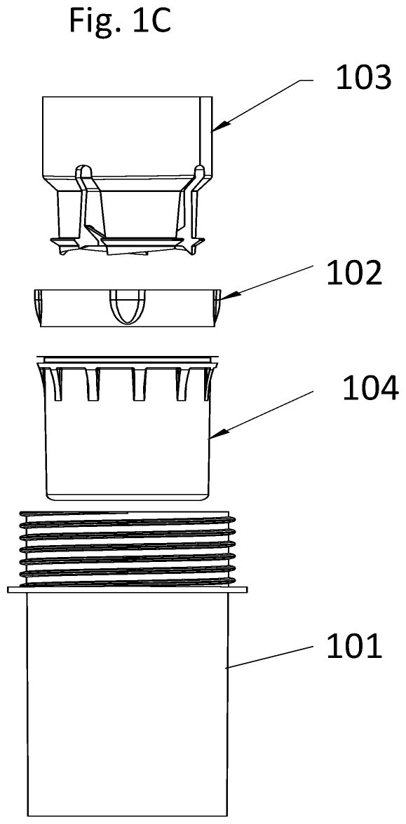

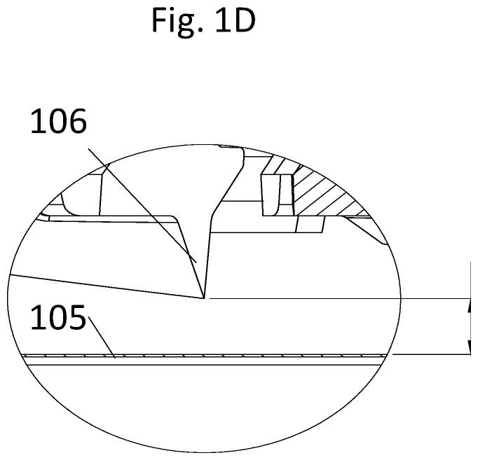

In some embodiments, as depicted in FIGS. 1A-C, the sample receptacle 101 contains a first chamber and a second chamber. In some embodiments, the second chamber contains a buffer cup 102. In some embodiments, the first chamber contains a displaceable shuttle 103. In some embodiments, as pictured in FIG. 1D, the displaceable shuttle comprises a piercing end 105 which is configured to be placed above the disruptable member 106 of the buffer cup.

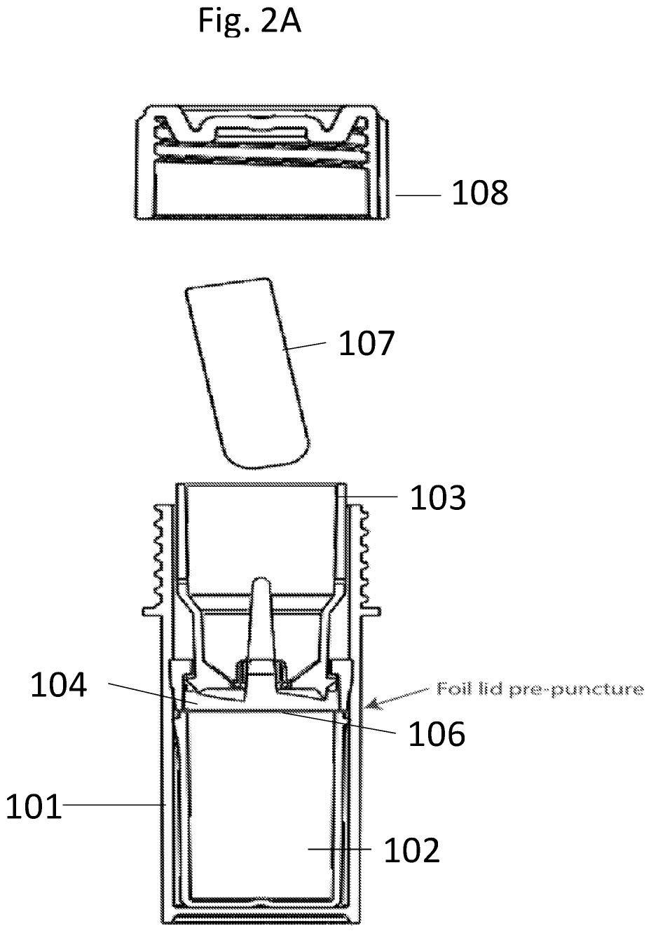

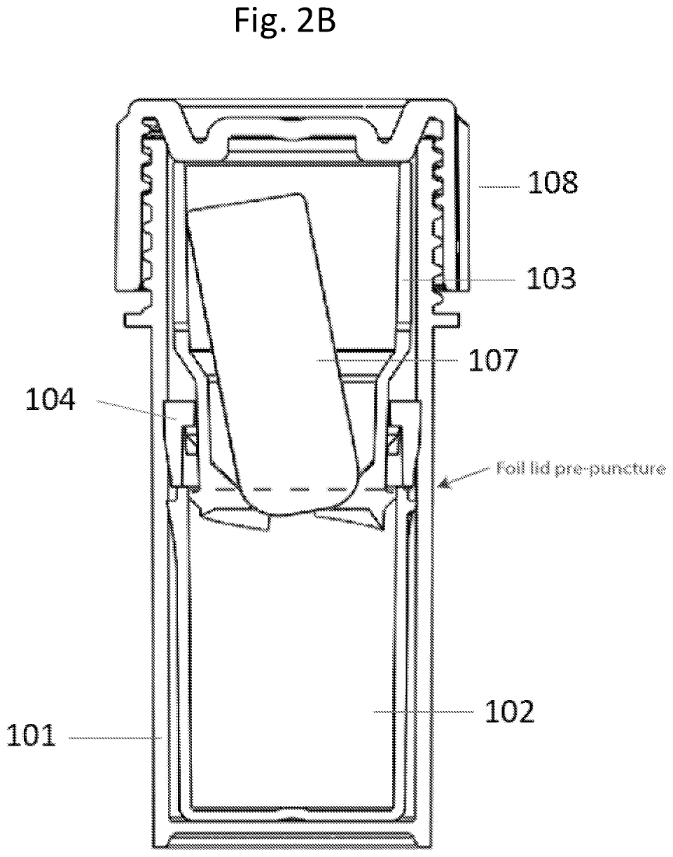

In some embodiments, as pictured in FIG. 2A, the sample receptacle 101 comprises an open end. In some embodiments, the sample collector 107 is placed within the displaceable shuttle. In some embodiments, the system comprises a cap 108. In some embodiments, as pictured in FIG. 2B, once the sample collector 107 is placed within the displaceable shuttle 103 of the sample receptacle 101, the cap 108 is used to seal the open end of the sample receptacle. In some embodiments, this causes the displaceable shuffle 103 to move towards the second chamber, disrupting the disruptable member and releasing the stabilization buffer so that the stabilization buffer within the buffer cup 102 is in fluid communication with the sample collector 107.

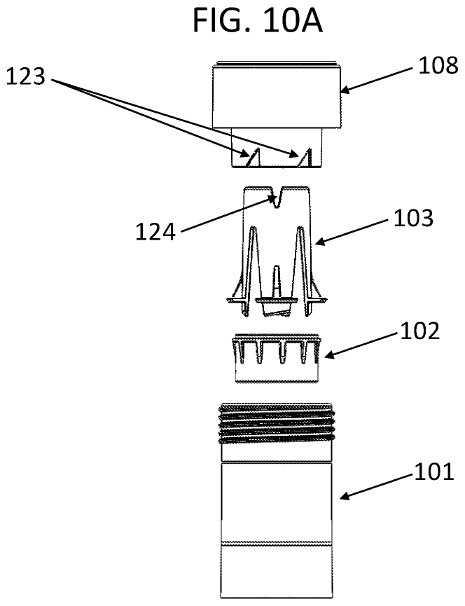

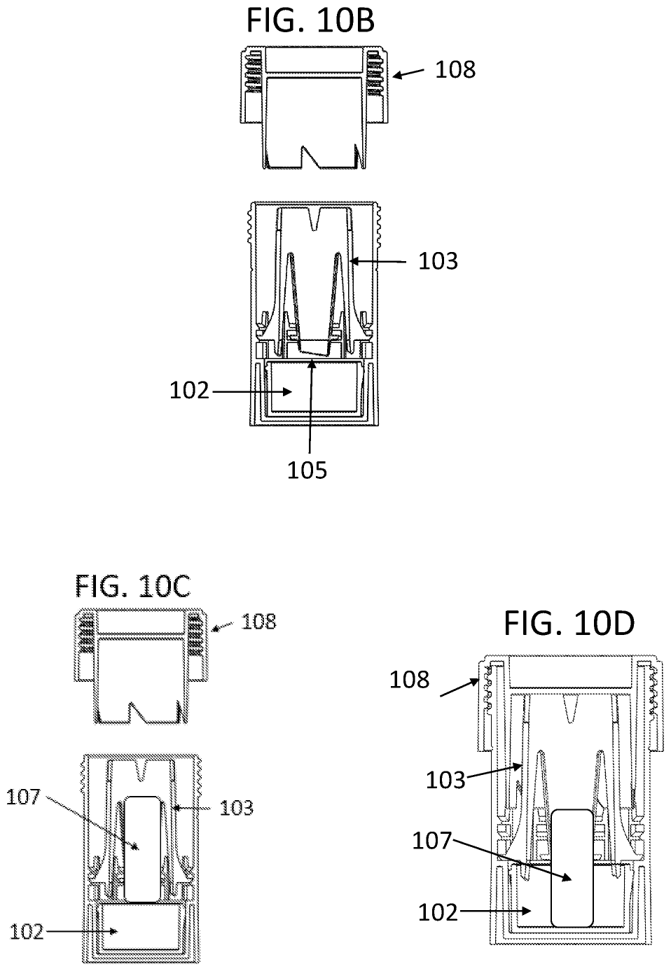

In some embodiments, as depicted in FIG. 10A the sample receptacle 101 comprises a cap 108, a displaceable shuttle 103, and a buffer cup 102. In some embodiments, the cap comprises a notch or a plurality of notches 123. In some embodiments, the displaceable shuttle comprises a notch or a plurality of notches 124. In some embodiments, as pictured in FIG. 10B-D, the sample receptacle contains a first chamber and a second chamber. In some embodiments, the second chamber contains a buffer cup 102. In some embodiments, the first chamber contains a displaceable shuttle 103. In some embodiments, as pictured in FIG. 10B, the displaceable shuttle comprises a piercing end 105 which is configured to be placed above the disruptable member of the buffer cup.

In some embodiments, as pictured in FIGS. 10A-D, the sample receptacle 101 comprises an open end. In some embodiments, as pictured in FIG. 10C, the sample collector 107 is placed within the displaceable shuttle. In some embodiments, the system comprises a cap 108. In some embodiments, as pictured in FIG. 10D, once the sample collector 107 is placed within the displaceable shuttle 103 of the sample receptacle 101, the cap 108 is used to seal the open end of the sample receptacle. In some embodiments, placing the cap on the sample receptacle causes the displaceable shuttle 103 to move towards the second chamber, disrupting the disruptable member and releasing the stabilization buffer so that the stabilization buffer within the buffer cup 102 is in fluid communication with the sample collector 107. In some embodiments, the sample collector 107 touches the bottom of the buffer cup 102.

In some embodiments, the cap 108 comprises a notch or a plurality of notches. In some embodiments, the displaceable shuttle 103 contains a notch or a plurality of notches. In some embodiments, the sample collector 107 is a tampon. In some embodiments, the tampon has a string. In some embodiments, the string of the tampon is caught in a notch or in a plurality of notches located on the displaceable shuttle. In some embodiments, the string of the tampon is caught in a notch or in a plurality of notches located on the cap. In some embodiments, the tampon string remains in a notch as the cap is screwed on to the sample receptacle. In some embodiments, the tampon string remains entirely within the sample receptacle as the cap is screwed on.

In some embodiments, systems and methods described herein comprise a sample receptacle. In some embodiments, the sample receptacle comprises a first chamber that is configured to hold the sample collector; a second chamber that is configured to contain a stabilization buffer; a disruptable member between the first chamber and the second chamber; and a piercer, wherein the piercer disrupts the disruptable member such that there is fluid communication between the first chamber and the second chamber. In some embodiments, the first chamber comprises an outer bottle and a piercer; wherein the piercer is configured to disrupt the disruptable member such that there is fluid communication between the first chamber and the second chamber. In some embodiments, the second chamber comprises a cap and a buffer cup, wherein the buffer cup comprises a stabilization buffer.

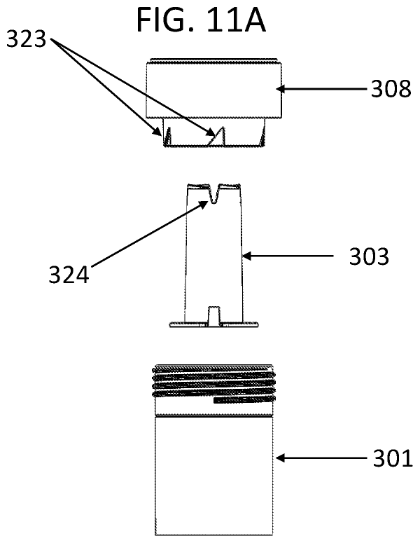

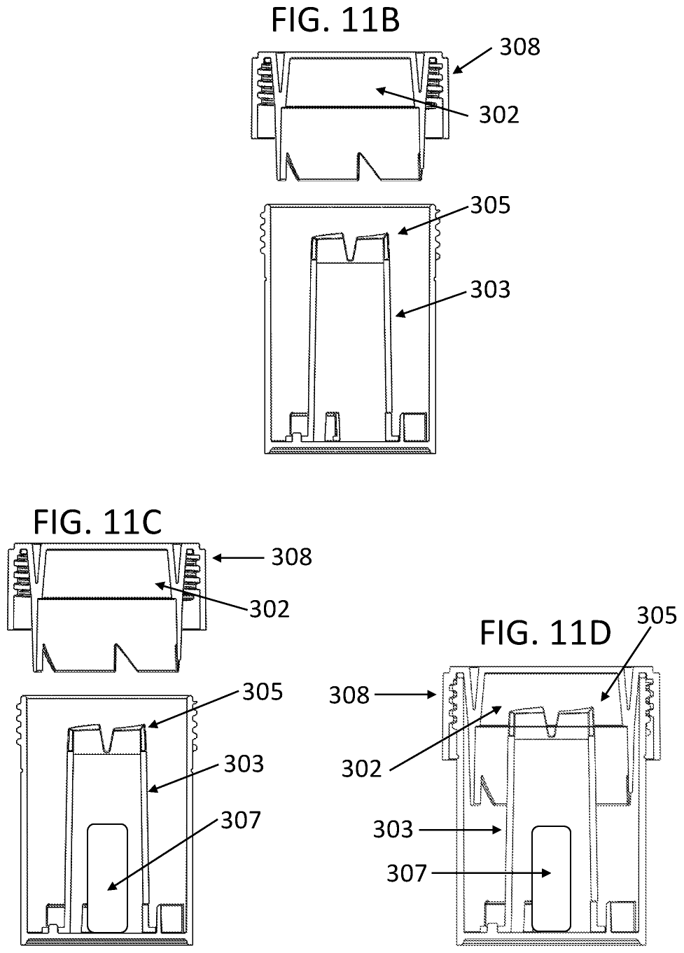

In some embodiments, as depicted in FIG. 11A the sample receptacle comprises a cap 308, a piercer 303, and an outer bottle 301. In some embodiments, the cap comprises a notch or a plurality of notches 323. In some embodiments, the piercer comprises a notch or a plurality of notches 324.

In some embodiments, as pictured in FIGS. 11B-C, the outer bottle 301 comprises an open end. In some embodiments, the outer bottle 301 contains a piercer 303. In some embodiments, the piercer is stationary. In some embodiments, the piercer 303 is connected to the outer bottle 301. In some embodiments, the piercer 303 is separate from the outer bottle 301. In some embodiments, as pictured in FIG. 11C, the sample collector 307 is placed within the piercer 303. In some embodiments, the piercer is a hollow cylinder. In some embodiments, the piercer comprises an open end. In some embodiments, the open end of the piercer is sliced into prongs. In some embodiments, as pictured in FIG. 11C, the sample collector 307 enters the piercer from the open end.

In some embodiments, as depicted in FIGS. 11B-D, the piercer 303 contains a piercing end 305. In some embodiments, the piercing end 305 is sharp. In some embodiments, the piercing end is angled and serves as a blade to pierce the buffer cup and expose the preservative buffer material to the sample collector.

In some embodiments, as pictured in FIGS. 11A-D, the system comprises a cap 308. In some embodiments, as pictured in FIGS. 11B-D, the cap 308 contains a buffer cup 302. In some embodiments, the buffer cup comprises a stabilization buffer. In some embodiments, the buffer cup 302 comprises a disruptable member. In some embodiments, the cap comprises a notch or a plurality of notches. In some embodiments, the piercer contains a notch or a plurality of notches. In some embodiments, the sample collector 307 is a tampon. In some embodiments, the tampon has a string. In some embodiments, the string of the tampon is caught in a notch or in a plurality of notches located on the piercer. In some embodiments, the string of the tampon is caught in a notch or in a plurality of notches located on the cap. In some embodiments, the tampon string remains in a notch as the cap is screwed on to the sample receptacle. In some embodiments, the tampon string remains entirely within the sample receptacle as the cap is screwed on.

In some embodiments, as pictured in FIG. 11D, once the sample collector 307 is placed within the piercer 303 of the outer bottle 301, the cap 308 is used to seal the open end of the outer bottle. In some embodiments, this causes the piercer 303 to disrupt a disruptable member so that the stabilization buffer within the buffer cup 302 is in fluid communication with the sample collector 307.

In some embodiments, the first chamber and the second chamber are continuous. In some embodiments, the sample receptacle comprises an open end. In some embodiments, the first chamber comprises an open end. In some embodiments, the sample receptacle is cylindrical.

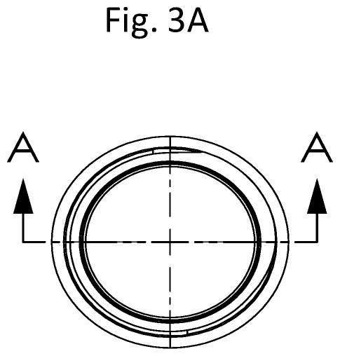

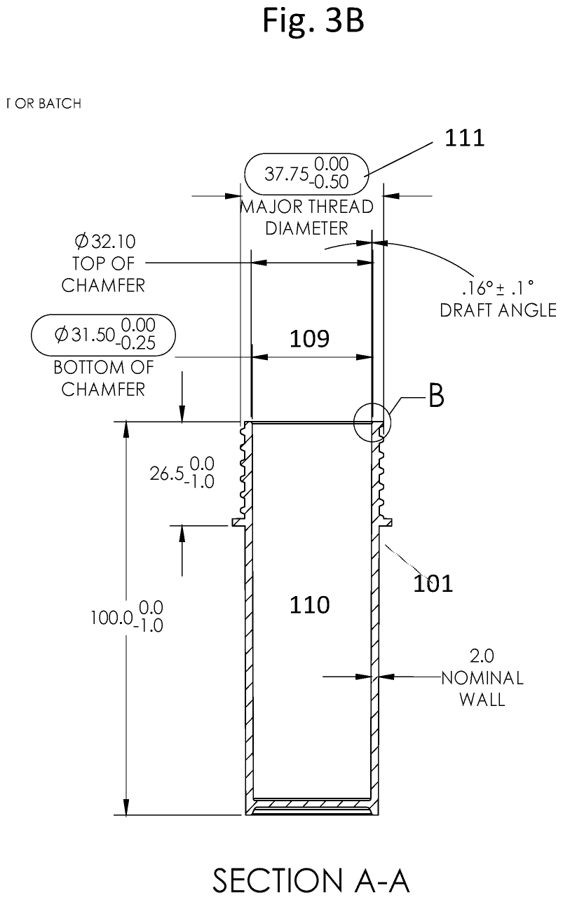

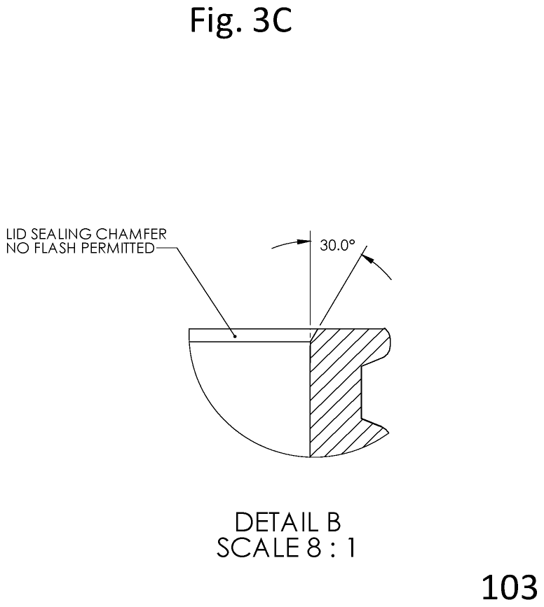

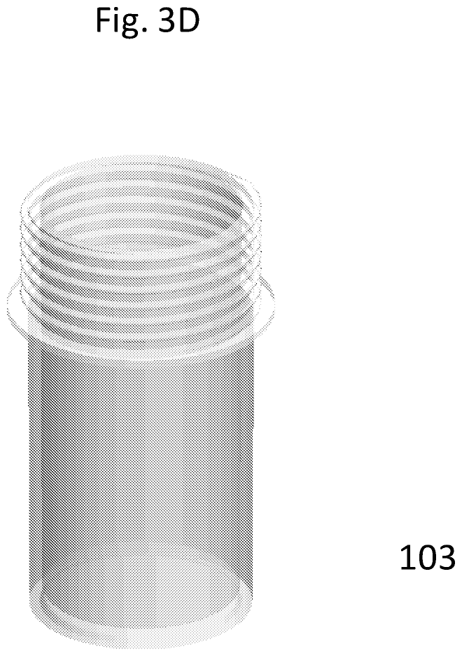

One example of the sample receptacle is depicted in FIG. 3A. A cross section is depicted in FIG. 3B. In some embodiments, the sample receptacle 101 comprises a first chamber 109 and a second chamber 110. In some embodiments, the first chamber comprises an open end 111. In some embodiments, the first chamber comprises threads to interface with the bottle cap. In some embodiments, the sample receptacle comprises a mechanical or visual indication for when a water-tight seal has been achieved. In some embodiments, the sampler receptacle comprises a pin or a lock and release mechanisms to prevent the displaceable shuttle from disrupting the disruptable membrane before the cap is placed on the sample receptacle. FIG. 3C depicts an inset of FIG. 3A. FIG. 3D depicts a three-dimensional version of one embodiment of a sample receptacle.

In some embodiments, placing a cap on the receptacle displaces the displaceable shuttle from the first detent on the locking ring to the second detent on the locking ring. In some embodiments, the cap is configured to engage the shuttle before fully closing. In some embodiments, the cap comprises a material that is softer than the receptacle. In some embodiments, the ridges of the displaceable shuttle are configured to prevent the displaceable shuttle from piercing the disruptable member before the cap is placed on the open end of the sample receptacle.

In some embodiments, placing a cap on the receptacle causes the piercer to contact the buffer cup. In some embodiments, the piercer is configured to avoid disrupting the disruptable membrane on the buffer cup before fully closing. In some embodiments, completely screwing a cap onto the receptacle causes the piercer to disrupt the disruptable member of the buffer cup. In some embodiments, the cap comprises a material that is softer than the receptacle.

In some embodiments, the cap comprises a cylindrical component with one open end. In some embodiments, the cap comprises internal threading. In some embodiments, the cap comprises external geometry for mechanical advantage while gripping and twisting. In some embodiments, the bottle cap is made of softer material than the bottle in order to create a watertight seal.

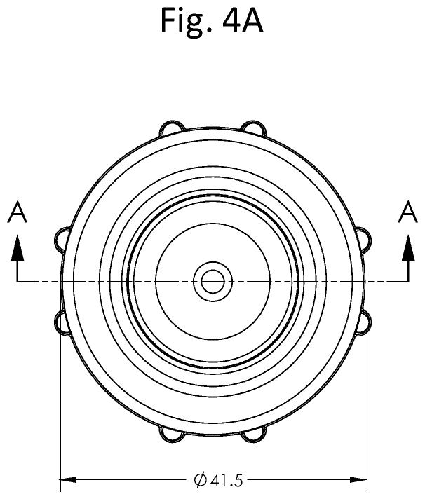

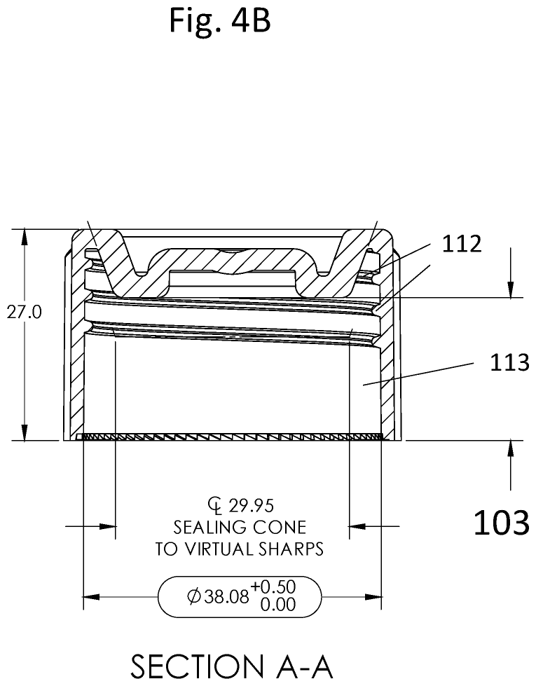

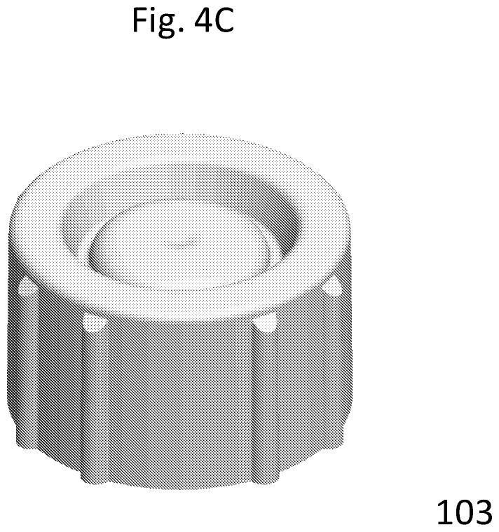

FIG. 4A depicts a top view of one embodiment of a cap. The cross section is depicted in FIG. 4B. In some embodiments, the internal threads 112 do not cover the entire height of the inner diameter of the cap. The non-threaded internal portion of the cap, hereby referred to as the skirt 113, is to guide the cap into proper thread alignment as the cap is placed over the extruding geometry of the shuttle as it sticks out of the bottle in the pre-use configuration. In some embodiments, the skirt allows the user to align the cap onto the bottle. This minimizes the event of cross threading as well as the need for the user to exert vertical force to push the shuttle down into the foil. In some embodiments, threading of the cap alone accomplishes the required vertical displacement of the shuttle for device activation. FIG. 4C depicts a three-dimensional schematic of the cap.

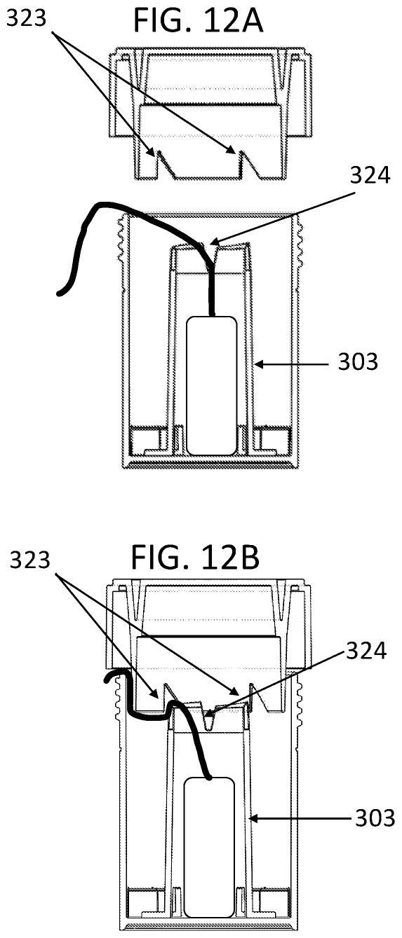

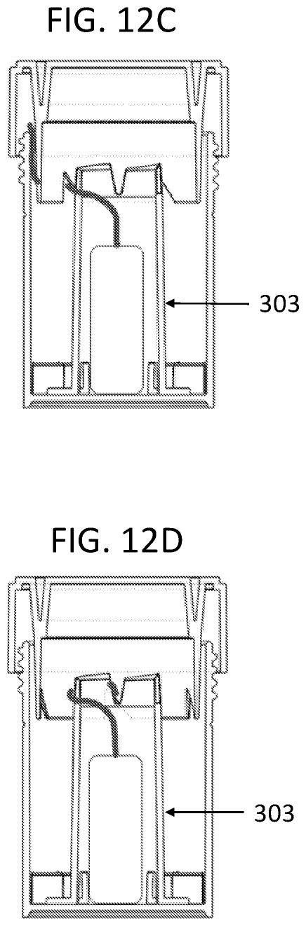

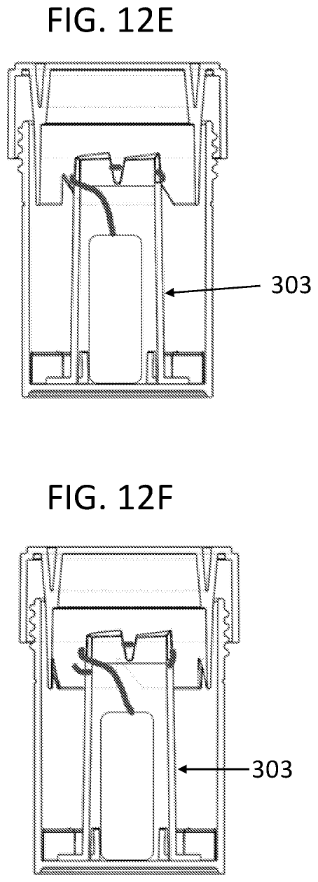

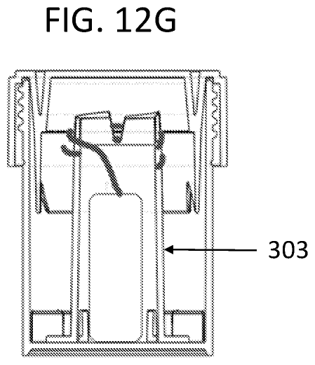

In some embodiments, as depicted in FIG. 12A the sample receptacle comprises a cap 308. In some embodiments, the cap comprises a notch or a plurality of notches 323. In some embodiments, the piercer comprises a notch or a plurality of notches 324. In some embodiments, the notches are V-shaped. In some embodiments, the sample collector 307 is a tampon. In some embodiments, the tampon has a string. In some embodiments, when the cap is placed on top of the sample receptacle, the tampon string is caught in a notch 324 located on the piercer or displaceable shuttle as depicted in FIG. 12A. As depicted in FIG. 12B, in some embodiments, as the cap is rotated, the tampon string is caught in another notch 323, located on the cap. In some embodiments, as the cap is screwed on to the sample receptacle, the tampon string remains caught in one or more notches. In some embodiments, as the cap is further screwed onto the sample receptacle, the tampon string becomes wrapped around either a shuttle or piercer 303 within the sample receptacle as depicted in FIGS. 12C-FIG. 12F. In some embodiments, when the cap is fully engaged, the tampon string is fully contained within the sample receptacle and does not disrupt the seal between the cap and the sample receptacle as depicted in FIG. 12G.

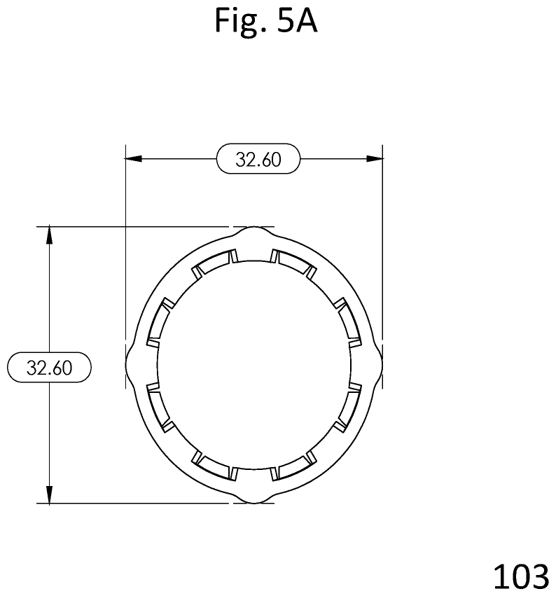

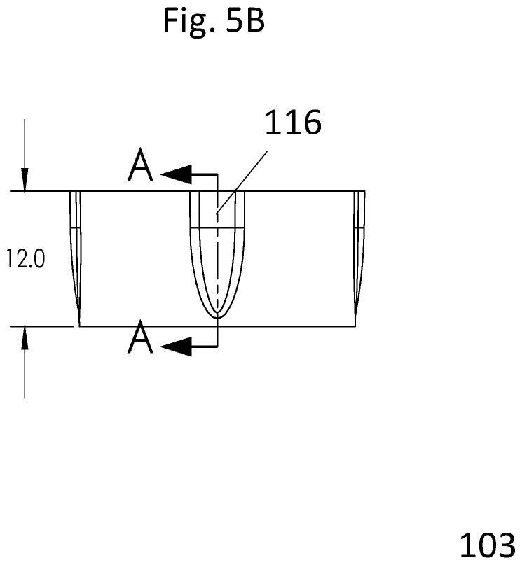

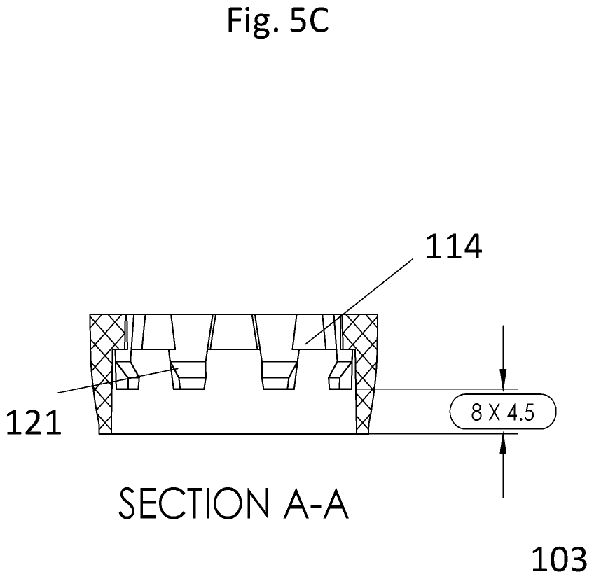

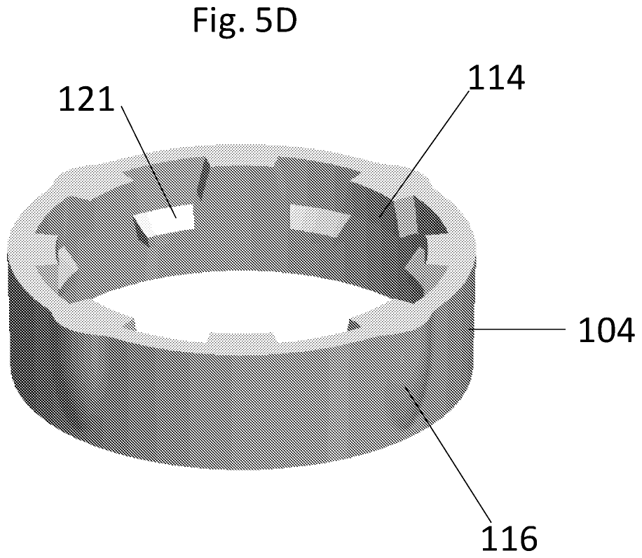

In some embodiments, the sample collector comprises a locking ring. FIG. 5A-5C depicts one embodiment of the locking ring. FIG. 5A depicts a top view. FIG. 5B depicts a front view of the locking ring. FIG. 5C depicts a cross-section of FIG. 5B. FIG. 5D depicts a three-dimensional schematic of a locking ring.

In some embodiments, the locking ring comprises at least two internal detents 114 and 121 which hold the displaceable shuttle in both pre-use configuration 114 and post-use configurations 121. In some embodiments, the pre-use configuration has the displaceable shuttle raised above the displaceable membrane. In some embodiments, the post-use configuration occurs after the cap is threaded onto the sample receptacle and the sharp inferior edges of the displaceable shuttle arms are translated vertically to puncture the buffer cup lid.

In one embodiment, the locking ring 104 is made a semi-compliant material with external nubs 116 that create an interference fit with the inner diameter of the sample receptacle.

In some embodiments, the inferior edge of the locking ring bottoms out on the top of the buffer cup. In some embodiments, the top of the buffer cup is also the disruptable member. In addition to the sealing process (heat, adhesive, ultrasonic welding, etc.), the bottomed-out locking ring provides extra support to the seal of the displaceable member.

In some embodiments, the displaceable shuttle is a dual-diameter hollow cylinder. In some embodiments, the displaceable shuttle comprises a first end and a second end. In some embodiments, the second end of the displaceable shuttle has a decreased diameter so that it can fit into the locking ring. In some embodiments, at the second end of the decreased diameter portion of the shuttle, there are snap-fit ridges. In some embodiments, the ridges interface with various detents in the locking ring and when snapped-in, maintain the vertical position of the shuttle. In some embodiments, the displaceable shuttle can rotate freely around its axis even when it is vertically constrained by snap-fitting into these detents.

In some embodiments, the second end of the shuttle is sliced into prongs. In some embodiments, the outer side of each prong has a snap-fit ridge to interface with the locking ring, and the inner side of each prong has a ledge. In some embodiments, when the sample collector is placed into the displaceable shuttle, this ledge holds the sample collector.

In some embodiments, the angled, bottom edge of the shuttle's prongs are sharp. In some embodiments, the bottom edge is angled and serves as a blade to pierce the buffer cup lid and expose the preservative buffer material to the sample collector.

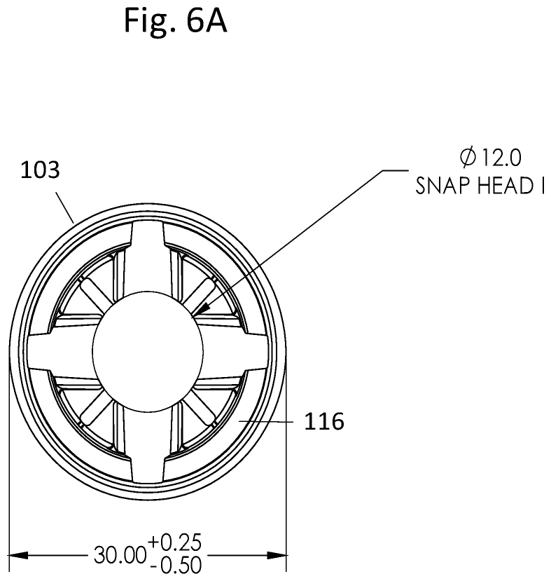

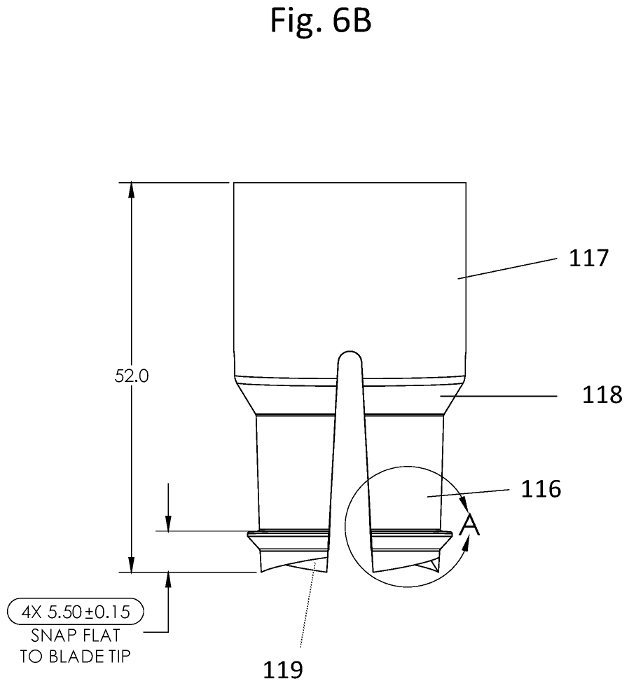

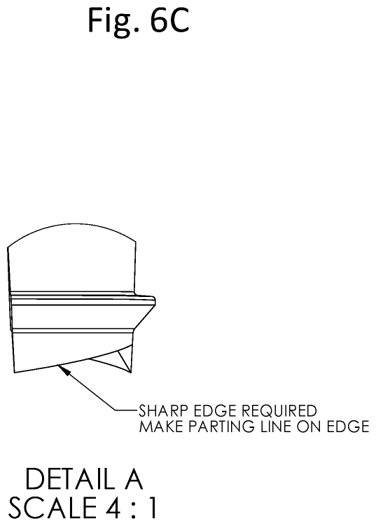

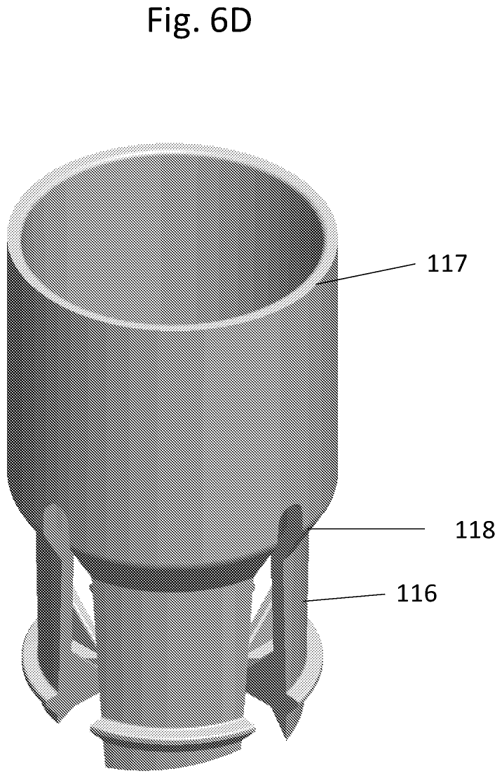

FIGS. 6A-6D depict one embodiment of the displaceable shuttle. FIG. 6A depicts a top view of a displaceable shuttle 103. In some embodiments, the displaceable shuttle comprises four prongs 116 to hold the sample collector. FIG. 6B depicts a side view of a displaceable shuttle. In some embodiments, the displaceable shuttle comprises a first end 117 and a second end 118, wherein the second end comprises prongs 116. In some embodiments, the prongs comprise a piercing end 119. FIG. 6C depicts a close view of a piercing end of a displaceable shuttle. FIG. 6D depicts a three-dimensional rendering of one embodiment of a displaceable shuttle.

In some embodiments, the second chamber comprises a buffer cup configured to contain the stabilization buffer. In some embodiments, the buffer cup comprises a cylinder with one open end. In some embodiments, the buffer cup comprises an outer ring and an inner ring, wherein the outer ring is larger in diameter than the inner ring. In some embodiments, the outer ring is larger in diameter and lower than the top ring. In some embodiments, the outer ring has an interference fit with the inner diameter of the bottle. In some embodiments, the outer ring has a ramped wedge to achieve its larger diameter, this ramped wedge is self-centering in manufacturing, as this component is pressed into (and bottoms out on this inside of) the bottle. In some embodiments, the interference fit creates a watertight seal so that buffer and sample are not trapped between the outside of the buffer cup and inside wall of the bottle when it comes time to extract the liquid sample from the device.

In some embodiments, there is an assembly vent which allows the buffer cup with its interference fit to be pressed into the bottle while mitigating pressure build up and force required for insertion. In some embodiments, the inner ring is the sealing surface to which the buffer cup lid is fused. In some embodiments, the fusion is accomplished by heat sealing, adhesive, ultrasonic welding, or other means of fusion.

In some embodiments, the disruptable member is attached to the buffer cup. In some embodiments, the disruptable member comprises a foil lid or a plastic film. In some embodiments, the disruptable member is made of pierceable, non-peelable foil. One method of manufacturing includes handling the disruptable member with vacuum tweezers to place it on top of the buffer cup after the buffer cup has been filled with the appropriate amount of buffer solution.

In some embodiments, the buffer cup is configured to contain the stabilization buffer. In some embodiments, the buffer cup is configured to hold at least about 10, 11, 12, 13, 14, 15, 16, 17, 18, 19, 20, 21, 22, 23, 24, 25, 26, 27, 28, 29, 30, or more than 30 milliliters of stabilization buffer. In some embodiments, the buffer cup comprises enough stabilization buffer to preserve at least about 1, 2, 3, 4, 5, 6, 7, 8, 9, or 10 milliliters of sample. In some embodiments, the ratio of stabilization buffer to sample is at least about 10:1, 10:2, 10:3, 10:4, 10:5, 10:6, 10:7, 10:8, 10:9 or 1:1. In some embodiments, the volume of buffer results in the sample being in constant contact with the stabilization buffer over an extended period of time. In some embodiments, the volume of stabilization buffer is sufficient to inhibit bacterial growth when compared to a sample combined with a lower volume of stabilization buffer.

In some embodiments, the material of both the buffer cup and the disruptable member are selected so they are not to interfere with the biological properties of the preservative buffer solution. In some embodiments, the disruptable member is thick and durable enough not to crease or fold while handling during manufacturing. In some embodiments, the disruptable member is also thick and durable enough not to rupture or burst while undergoing pressure differentials seen from temperature changes or shipping conditions. In some embodiments, the disruptable member is pierceable and has crack-propagation characteristics that allow the sharp edges of the shuttle to pierce holes through which buffer solution can easily drain.

In some embodiments, the diameter of the disruptable member is greater than the “inner ring” on the buffer cup (the surface on which disruptable member is sealed to the buffer cup), to allow for tolerance of placement during manufacturing. In some embodiments, the buffer cup is filled with a stabilization buffer then the disruptable member is heat sealed, fused, adhesive, or cured to the top sealing surface.

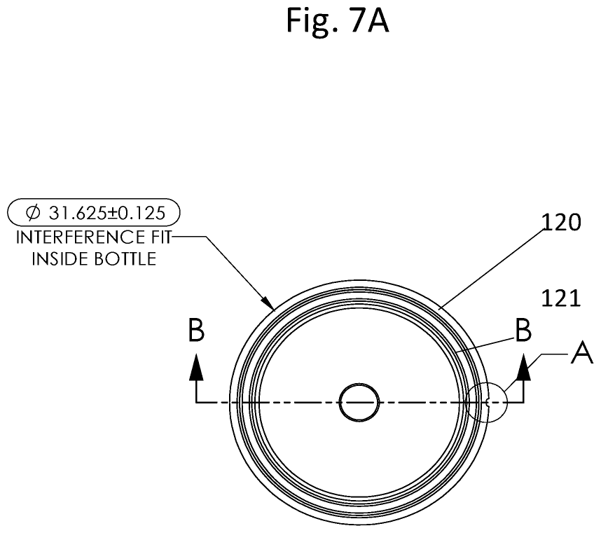

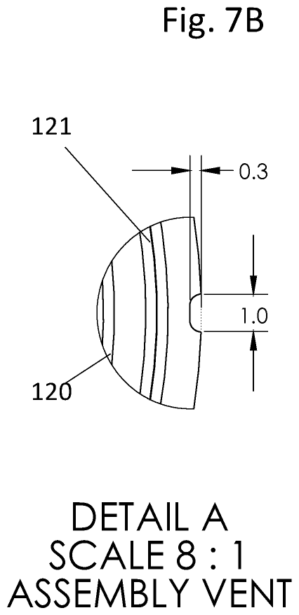

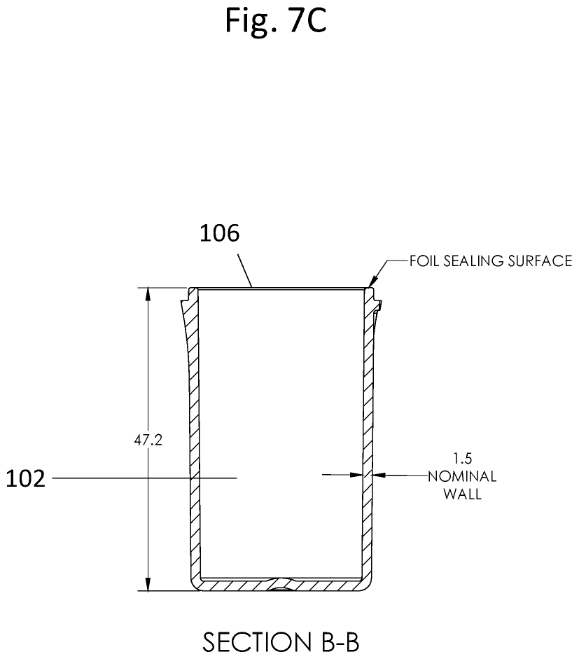

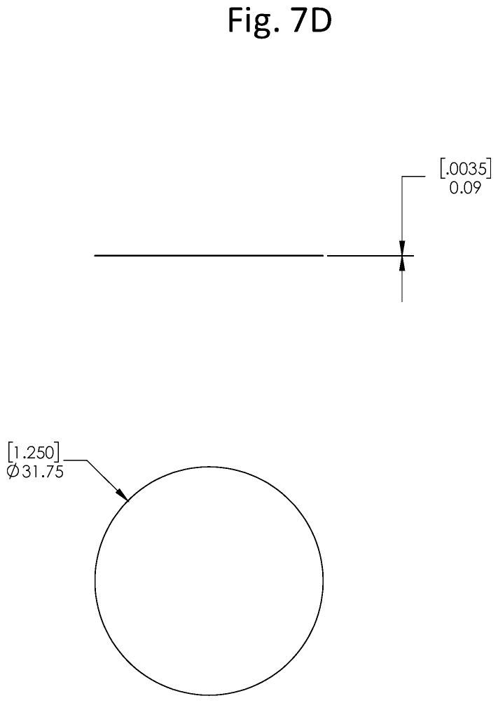

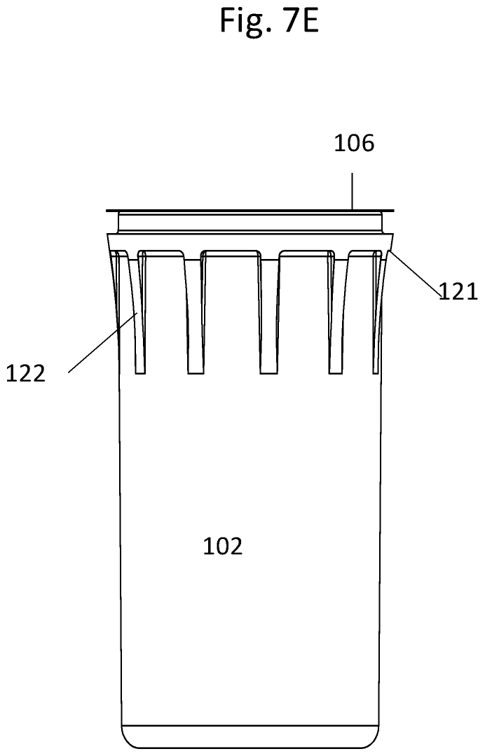

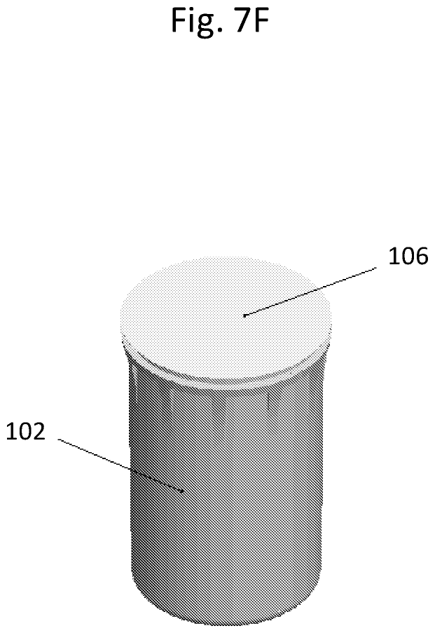

One embodiment of the buffer cup is depicted in FIGS. 7A-7F. FIG. 7A depicts a top view of a buffer cup comprising an inner ring 120 and an outer ring 121. FIG. 7B depicts an inset of FIG. 7A showing the inner ring 120 and the outer ring 121. FIG. 7C depicts a cross section of a buffer cup 102 and a disruptable member 106. FIG. 7D depicts a cross section and top view of the disruptable member. FIG. 7E depicts a side view of a buffer cup 102. In some embodiments, the disruptable member 106 is sealed to the top of the buffer cup 102. In some embodiments, the disruptable member 106 is sealed to the bottom of the buffer cup 102. In some embodiments the outer circle 121 is wider than the circumference of the rest of the buffer cup. In some embodiments, the buffer cup comprises ridges 121 continuous with the outer circle. FIG. 7F depicts a three-dimensional rendering of a buffer cup 102 with a disruptable member 106.

In some embodiments, the displaceable shuttle is configured to hold the sample collector within the first chamber. In some embodiments, the sample collector is not compressed. In some embodiments, the sample collector is not compressed when the sample receptacle is closed. In some embodiments, the displaceable shuttle is configured such that it does not compress the sample collector. In some embodiments, the sample receptacle comprises a compression mechanism configured to compress the sample collector. In some embodiments, the sample receptacle does not comprise a compression mechanism configured to compress the sample collector.

In some embodiments, upon disruption of the disruptable member, the system is configured to retain the stabilization buffer between the first chamber and the second chamber when the device is rotated, placed horizontally, and/or inverted from its vertical position. In some embodiments, the sample receptacle is configured to be inverted or agitated during use and shipment so that the buffer solution sufficiently coats the tampon which contains harvested human cells. In some embodiments, upon rotation, horizontal placement, or inversion, the device is configured such that the stabilization buffer washes over the sample collector thereby releasing the biological sample from the sample collector into the stabilization buffer. In some embodiments, the biological sample is released from the sample collector into the stabilization buffer.

In some embodiments, the preservation solution includes Biomatrica LBgard® or Biomatrica RNAgard®. In some embodiments, the preservation solution preserves RNA at room temperature for at least 1, 2, 3, 4, 5, 6, 7, 14, or 21 days. In some embodiments, the preservation solution prevents degradation of at least 50%, 60%, 70%, or 80% of the RNA. In some embodiments, the pH range of the preservation solution is from pH 3 to pH 8, or more preferably from pH 3 to pH 6.5. In some embodiments, the preservation solution preserves DNA at room temperature for at least 1, 2, 3, 4, 5, 6, 7, 14, or 21 days. In some embodiments, the preservation solution prevents degradation of at least 50%, 60%, 70%, or 80% of the DNA. In some embodiments, the pH range of the preservation solution is from pH 5 to pH 10, or more preferably from pH 6 to pH 9.

In some embodiments of methods and systems provided herein, the preservation solution preserves the nucleic acid at room temperature for at least 1, 2, 3, 4, 5, 6, 7, 14, or 21 days. In some embodiments, the preservation solution prevents degradation of at least 50%, 60%, 70%, or 80% of the nucleic acid. In some embodiments, the pH range of the preservation solution is from pH 3 to pH 8, or more preferably from pH 3 to pH 6.5. In some embodiments, the preservation solution preserves RNA at room temperature for at least 1, 2, 3, 4, 5, 6, 7, 14, or 21 days. In some embodiments, the preservation solution prevents degradation of at least 50%, 60%, 70%, or 80% of the RNA. In some embodiments, the pH range of the preservation solution is from pH 3 to pH 8, or more preferably from pH 3 to pH 6.5. In some embodiments, the preservation solution preserves DNA at room temperature for at least 1, 2, 3, 4, 5, 6, 7, 14, or 21 days. In some embodiments, the preservation solution prevents degradation of at least 50%, 60%, 70%, or 80% of the DNA. In some embodiments, the pH range of the preservation solution is from pH 5 to pH or more preferably from pH 6 to pH 9. In some embodiments, the preservation solution includes Biomatrica LBgard® or Biomatrica RNAgard®.

In some embodiments, the preservation solution comprises a spike-in. As used herein, a “spike-in” is a molecule, such as a nucleic acid, a cell, or a set of molecules or cells added to a sample, wherein the spike-in is used to quantitatively or qualitatively assess or to normalize a sample. In some embodiments, the spike-in comprises a nucleic acid spike-in. In some embodiments, the nucleic acid spike-in comprises a DNA spike-in, an RNA spike-in, a bacterial spike-in, or a combination thereof. In some embodiments, the DNA spike-in comprises a synthetic DNA or a plurality of synthetic DNAs. In some embodiments, the RNA spike-in comprises a synthetic RNA or a plurality of synthetic RNAs. In some embodiments, the RNA spike-in comprises a set of RNA transcripts developed by the External RNA Controls Consortium (ERCC).

In some embodiments, the preservation solution comprises a mucolytic agent. In some embodiments, the mucolytic agent dissociates (e.g., “unclump”) at least a portion of cellular aggregations in the cervicovaginal sample. In some embodiments, the mucolytic comprises acetylcysteine, ambroxol, bromhexine, carbocisteine, domiodol, dornase alfa, eprazinone, erdosteine, letosteine, mannitol, mesna, neltenexine, sobrerol, stepronin, tiopronin, N-acetyl-L-cysteine, L-acetyl cysteine/Liberase™, or a combination thereof.

In some embodiments, the preservation solution comprises an expectorant. In some embodiments, the expectorant comprises althea root, antimony pentasulfide, creosote, guaiacolsulfonate, guaifenesin (+oxomemazine), ipecacuanha, levoverbenone, potassium iodide, senega, tyloxapol, ammonium chloride, or a combination thereof.

In some embodiments, the preservation solution comprises a surfactant. In some embodiments, the surfactant comprises polyoxyethylene glycol octylphenol ethers; polyoxyethylene glycol alkylphenol ethers; polyoxyethylene glycol sorbitan alkyl esters; sorbitan alkyl esters; polyethylene glycol; polypropylene glycol; carboxylates; sulphonates; petroleum sulphonates; alkylbenzenesulphonates; naphthalenesulphonates; olefin sulphonates; alkyl sulphates; sulphates; sulphated esters; sulphated alkanolamides; alkylphenols; ethoxylated aliphatic alcohol; polyoxyethylene surfactants; carboxylic esters; polyethylene glycol esters; anhydrosorbitol esters; glycol esters; carboxylic amide; monoalkanolamine condensates; polyoxyethylene fatty acid amides; quaternary ammonium salts; polyoxyethylene alkyl and alicyclic amines; N,N,N′,N′ tetrakis substituted ethylenediamines; 2-alkyl 1-hydroxethyl 2-imidazolines; or a combination thereof.

In some embodiments, the preservation solution comprises a nuclease. In some embodiments, the nuclease comprises a Benzonase®, DNase I, DNase II, Exonuclease III, Micrococcal Nuclease, Nuclease P1, Nuclease Si, Phosphodiesterase I, Phosphodiesterase II, RNase A, RNase H, RNase Ti, or a combination thereof.

In some embodiments, the preservation solution comprises a protease. In some embodiments, the protease comprises adispase II, trypsin, pronase, collagenase 1, collagenase 2, collagenase 3, collagenase 4, hyaluronidase, pepsin, papain, chemotrypsin, chymase, clostripain, complement C1r, complement C1s, complement factor D, complement factor I, cucumisin, dipeptidyl peptidase, elastase, endoproteinase, enterokinase, Factor X Activated, caspase, cathepsin, matrix metalloprotease, or a combination thereof.

In some embodiments, the osmolality of the preservation solution comprises from about 310 to about 410 mOsm kg₋₁. In some embodiments, the osmolality of the preservation solution comprises from about 95 to about 210 mOsm kg₋₁.

In some embodiments, the preservation solution does not comprise a fixative. In some embodiments, the fixative comprises an alcohol, an aldehyde, an oxidizing agent, a metallic fixative or a combination thereof. In some embodiments, the alcohol comprises methanol, ethanol, propanol, isopropanol, butanol, or a combination thereof. In some embodiments, the aldehyde comprises formaldehyde, glutaraldehyde, or a combination thereof. In some embodiments, the oxidizing agent comprises an osmium tetraoxide, potassium permanganate, potassium dichromate, or a combination thereof. In some embodiments, the metallic fixative comprises a mercuric chloride, a picric acid, or a combination thereof. In some embodiments, the preservation solution does not comprise an alcohol, an aldehyde, an oxidizing agent, a metallic fixative, or a combination thereof.

In some embodiments, the preservation solution comprises a binding agent. In some embodiments, the binding agent selectively binds to an target cell or a non-target cell of the individual. In some embodiments, the target cell comprises an endothelial cell, an epithelial cell, a leukocyte, a mesenchymal cell, or a combination thereof. In some embodiments, the non-target cell comprises an endothelial cell, an epithelial cell, a leukocyte, a mesenchymal cell, spermatozoa, bacterial cell, fungal cell, or a combination thereof. In some embodiments, the non-target cell comprises different than the target cell. In some embodiments, the binding agent selectively binds to at least one protein or fragment thereof. In some embodiments, the at least one protein or fragment thereof comprises a biomarker of endometriosis. In some embodiments, the binding agent selectively binds to a nucleic acid. In some embodiments, the nucleic acid comprises a biomarker of endometriosis. In some embodiments, the binding agent is immobilized, for example, to a bead or to a surface of a component of the systems described herein. In some embodiments, the binding agent is coupled to the bead or the surface of the system. In some embodiments, the binding agent is reversibly or irreversibly coupled to the bead or the surface of the system. In some embodiments, the binding agent comprises a cleavable moiety, for example, a cleavable linker. In some embodiments, the cleavable linker is cleaved photolytically, chemically, thermally, or enzymatically.

In some embodiments, from 0.1 ml to 0.9 ml, from 0.3 ml to 0.7 ml, or from 0.4 ml to 0.6 ml of preservation solution is diluted to form a diluted preservation solution. In some embodiments, the preservation solution comprises Biomatrica LBgard® or Biomatrica RNAgard®. In some embodiments, the preservation solution is diluted in from 4.5 ml to 12.5 ml, from 6.5 ml to 10.5 ml or from 7.5 ml to 9.5 ml of a second solution. In some embodiments, the second solution is distilled water. In some embodiments, a diluted preservation solution is used in the methods and/or systems provided herein. In some embodiments, the diluted preservation solution is added to a sample collector at from 2 ml to 6 ml or from 3 ml to 5 ml of diluted preservation solution per gram of fluid that is absorbed into the sample collector. In some embodiments, a sample collector absorbs up to 6 g of fluid, thus, about 18 ml to about 30 ml of diluted preservation solution is added to the light absorbency tampon. In some embodiments, the diluted preservation solution is added to the sample collector in the system described herein, following the rupture of the disruptable member. Accordingly, as the absorbency of the sample collector increases, the amount of diluted preservation solution to be added increases.

In other embodiments, the preservation solution is not diluted. In some embodiments, the undiluted preservation solution is used in the methods and/or systems provided herein. In some embodiments, the undiluted preservation solution is added to a sample collector at about 3 ml to about 5 ml of undiluted preservation solution per gram of fluid that is absorbed into the sample collector. In some embodiments, a light absorbency tampon absorbs up to 6 g of fluid, thus, about 18 ml to about 30 ml of undiluted preservation solution is added to the sample collector. In some embodiments, the undiluted preservation solution is added to the sample collector in the system described herein, following the rupture of the disruptable member. Accordingly, as the absorbency of the sample collector increases, the amount of undiluted preservation solution to be added increases.

B. Sample Collector

In some embodiments, the system described herein comprises a sample collector that collects and retains the biological sample from a vaginal canal of the subject.

In some embodiments, a sample, such as a menstrual fluid sample or a sample of another fluid, is collected from a subject using a sample collector which collects fluid from the vaginal cavity. In some embodiments, the sample collector is insertable in the vaginal canal. In some embodiments, a sample collector is placed in the vagina or outside the vagina for sample collection. In some embodiments, a sample collector collects a sample by pooling, holding, catching, directing, or absorbing the sample. In some embodiments, a sample collector is absorbent, semi-absorbent, or non-absorbent. In some embodiments, a sample collector is soluble in a buffer. In some embodiments, a sample collector is broken down, for example by exposing the sample collector to an acidic environment, a basic environment, or an enzyme. In some embodiments, sample collectors comprise a pad, a tampon, a vaginal cup, a cervical cap, a menstrual disk, a cervical disk, a sponge, a plug, a swab, or an interlabial pad. In some embodiments, the sample collector is a tampon. In some embodiments, a tampon has a string. In some embodiments, more than one type of sample collector are used.

In some embodiments, the sample collected from the vaginal cavity is a biological matrix comprising a plurality of cell types. In some instances, the cell types include immune cells, ovarian cells, fallopian tube cells, endometrial cells, and cervical cells. In some embodiments, the fluid is menstrual fluid, including menstrual blood.

In some embodiments, the sample collector comprises an absorbent-diffuse material that collects, retains or releases the biological material. In some embodiments, the absorbent-diffuse material comprises one or more of a plant fiber material, a disposable material, a flushable material, a biodegradable material, and an organic material. In some embodiments, a sample collector is disposable. In some embodiments, a disposable sample collector is discarded or broken down after use. In some embodiments, a disposable sample collector is dissolvable, biodegradable, recyclable, or compostable. Typically, one disposable sample collector is used to collect one sample from one subject. In some embodiments, a sample collector is reusable. In some embodiments, a reusable sample collector is washable, sterilizable, or autoclavable. In some embodiments, a reusable sample collector is resistant to degradation, tearing, pore formation, or dissolution. In some embodiments, a reusable sample collector comprises anti-microbial, antibacterial, antiviral, or antifungal properties. In some embodiments, a reusable sample collector is used one or more times to collect one or more samples. In some embodiments, a reusable sample collector is used about 1, 2, 3, 4, 5, 6, 7, 8, 9, 10, 15, 20, 25, 30, or more times to collect one or more fluid samples. In some embodiments, a reusable sample collector is used to repeatedly collect fluid samples from one subject. In some embodiments, a reusable sample collector is used to collect samples from a plurality of subjects.

In some embodiments, samples are collected outside the menstrual window, e.g., between the time of the subject's periods. In such cases, a non-menstrual fluid is collected using the sample collector. In some embodiments, non-menstrual fluid which is collected include vaginal secretions, cervical mucus, cervicovaginal fluid, spotting blood (i.e., from between periods), amniotic fluid, a mucus plug, or other vaginal discharge. In some embodiments, non-menstrual fluid is collected and analyzed using a protocol which is used to collect and analyze menstrual fluid.

In some embodiments, such methods comprise collection of a sample of fluid from the vaginal cavity of a subject. In some embodiments, collection is, for example, performed using a sample collector such as a sponge, a tampon, a pad, or another absorbent material. In some embodiments, a further description of sample collectors and methods is provided herein.

In some embodiments, the stabilization buffer comprises a preservative. In some embodiments, the stabilization buffer is formulated to preserve one or more of intact cells, nucleic acids, DNA, RNA, protein or metabolite(s). In some embodiments, the system is configured to preserve one or more of more of intact cells, nucleic acids, DNA, RNA, protein or metabolite(s) for at least 72 hours at room temperature. In some embodiments, stabilization comprises adding the sample to a buffer, such as a preservation buffer. In some embodiments, stabilization also comprises storage at a specific temperature (e.g., 4° C., −20° C., room temperature, or another acceptable temperature, including those described herein). In some embodiments, stabilization comprises moving the sample to a new vessel and placing a lid or covering on the vessel. In some embodiments, the vessel is vacuum sealed or the sample is stored under argon gas or nitrogen gas.

In some instances, at least 20%, at least 30%, at least 40%, at least 50%, at least 60%, at least 70%, at least 80%, or at least 90% of the target cells in the sample are intact. In some embodiments, the target cells is endometrial cells. In some embodiments, the target cells is endothelial cells, epithelial cells, leukocytes, mesenchymal cells, or a combination thereof. In some instances, at least 95% of the target cells in the sample are intact. An intact cell is a cell which does not have a ruptured cell membrane. An intact cell is a cell in its native state. An intact cell is a viable cell, wherein the viable cell is cultured in a cell culture.

In some instances, at least 20%, at least 30%, at least 40%, at least 50%, at least 60%, at least 70%, at least 80%, or at least 90% of the target cells in the sample are viable. In some embodiments, the term “viable” means intact, living, and/or capable of proliferation. Viability of a plurality of cells is assessed by measuring membrane permeability, enzymatic activity, metabolic activity, DNA synthesis, membrane potential, proliferation marker expression, or a combination thereof.

In some embodiments, preservation and stabilization by such methods preserves a component of the sample, such that the component remains substantially unchanged or that the component does not deteriorate significantly while the sample is stored. In some embodiments, components include a cell, a nucleic acid, a gene target biomarker, a protein biomarker, another protein, a microorganism (e.g., a pathogen or member of a microbiome), a small molecule, a metabolite, or another component.

C. Harvester

In some embodiments, described herein is a harvester mechanism.

In some embodiments, the harvester mechanism comprises a handle and a stem, wherein the stem is configured to be inserted into the sample receptacle and to compress the sample collector. In some embodiments, the harvester mechanism comprises teeth 205 that interface with the sample collector. In some embodiments, the teeth of the harvester are configured to engage the lip on the inside of the displaceable shuttle or piercer to retain the sample collector within the displaceable shuttle or piercer. In some embodiments, the handle of the harvester mechanism is configured to provide a mechanical advantage when compressing the sample collector.

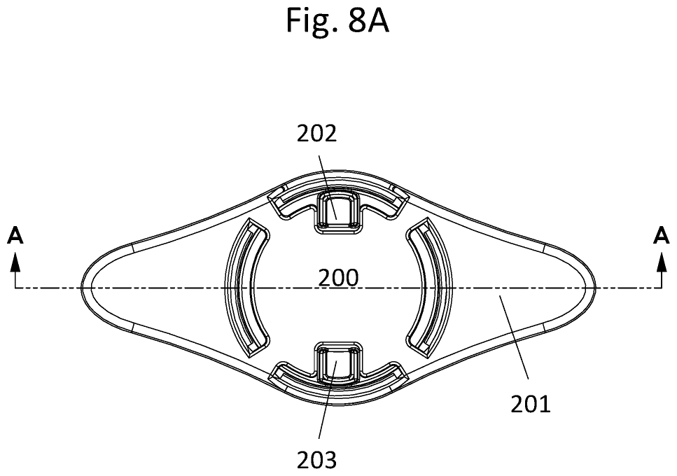

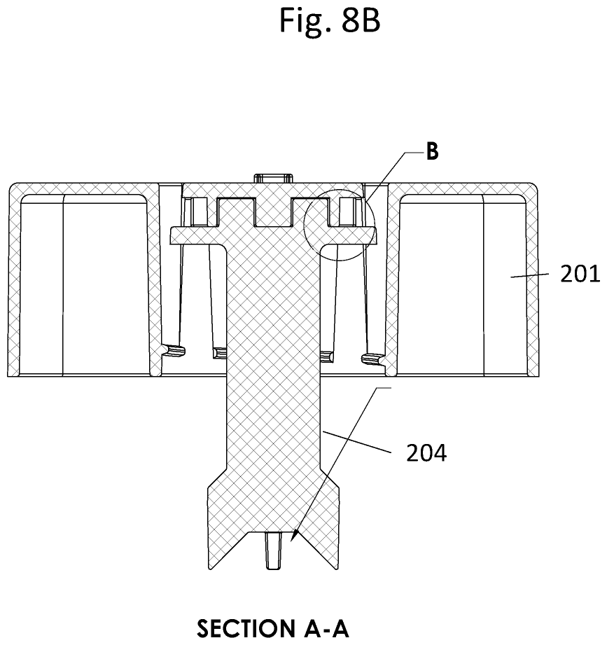

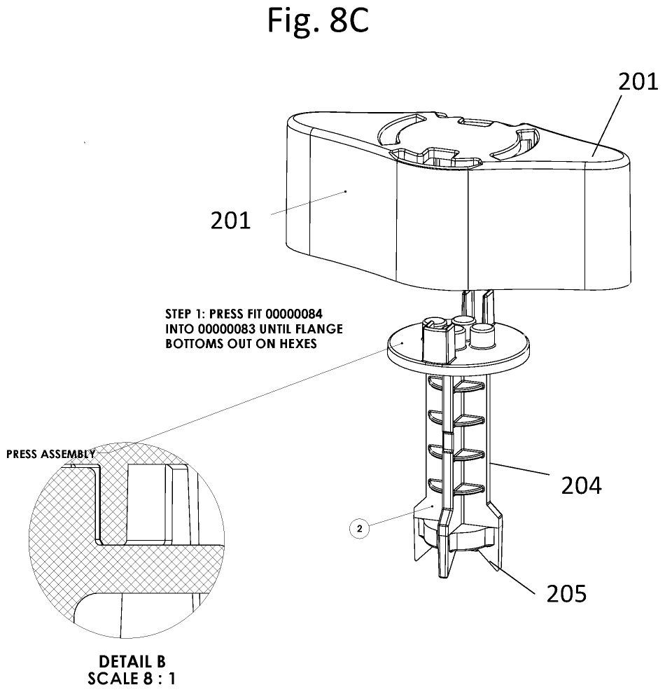

In some embodiments, the harvester comprises the device pictured in FIGS. 8A-8C. FIG. 8A depicts a top view of the harvester 200. The harvester comprises two wings 201, a fluid passageway 202 and an air passageway 203. FIG. 8B depicts a cross section of the harvester comprising two wings 201 and a stem 204. FIG. 8C depicts a detailed inset of the cross section as well as a side view of the harvester comprising a stem 204 and two wings 201.

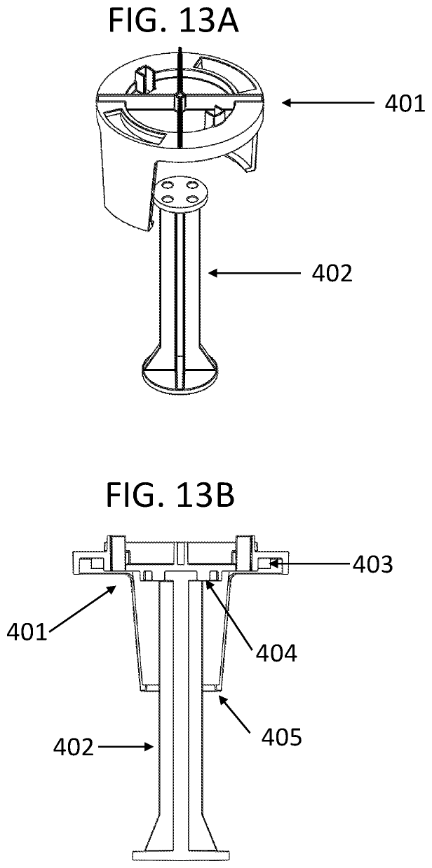

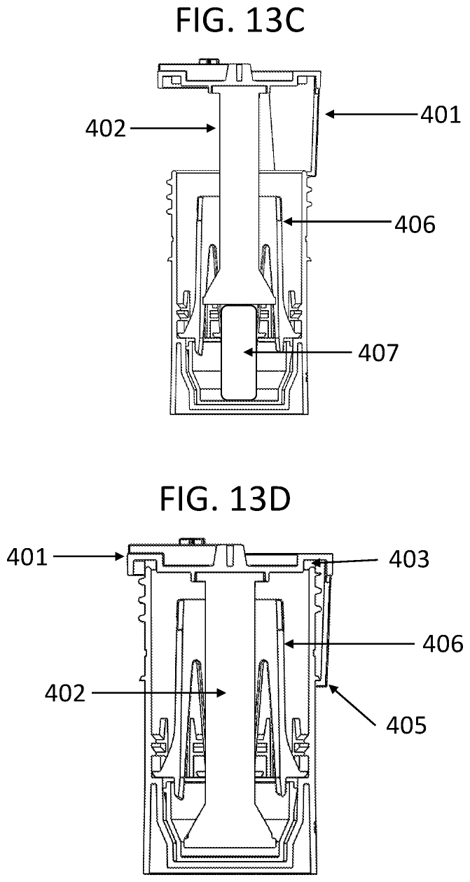

In some embodiments, the harvester comprises the device pictured in FIGS. 13A-13D. FIG. 13A depicts the harvester cap 401 and the stem 402. As depicted in FIG. 13B, the harvester may comprise a rubber seal 403, a press-fit pin 404, and snap arms 405. In some embodiments, a press-fit pin 404 is used to assemble the cap and the stem together.

In some embodiments, as pictured in FIG. 13C and FIG. 13D, the harvester compresses the sample collector 407. In some embodiments, the stem 402 is configured to be inserted into the sample receptacle and to compress the sample collector 407. In some embodiments, the harvester comprises snap arms 405 that interface with the sample collector. In some embodiments, the snap arms of the harvester are configured to engage a lip on the sample receptacle to retain the sample collector within the displaceable shuttle or piercer 406. In some embodiments, the harvester comprises a rubber seal 403 configured to prevent unwanted leakage of sample. In some embodiments, the harvester is pressed onto the bottle with an arbor press. In some embodiments, a mechanical advantage is provided by the arbor press.

In some embodiments, the harvester mechanism comprises a fluid passageway configured to allow the stabilization buffer and the biological sample to be moved from the second chamber of the sample receptacle into a second receptacle. In some embodiments, the harvester comprises an air passageway that is separate from the fluid passageway. In some embodiments, the fluid passageway is selected from the group consisting of a spout, a luer lock, a syringe port, and a one-way valve.

In some embodiments, the harvester comprises the device pictured in FIGS. 8A-8C. FIG. 8A depicts a top view of the harvester 200. The harvester comprises two wings 201, a fluid passageway 202 and an air passageway 203. FIG. 8B depicts a cross section of the harvester comprising two wings 201 and a stem 204. FIG. 8C depicts a detailed inset of the cross section as well as a side view of the harvester comprising a stem 204 and two wings 201.

### II. Methods of Use

In some embodiments, described herein is a method of using the sample receptacles described herein. In some embodiments, described herein is a method of collecting a cervicovaginal sample, the method comprising: collecting a cervicovaginal sample with a sample collector; placing the sample collector within the sample receptacle disclosed herein; and closing the sample receptacle, such that the displaceable shuttle pierces the disruptable member.

In some embodiments, a sample collector is left in place for a pre-determined amount of time to collect a fluid sample. In some embodiments, at least 5 minutes, 10 minutes, 15 minutes, 30 minutes, 45 minutes, 1 hour, 1.5 hours, 2 hours, 3 hours, 4 hours, 5 hours, 6 hours, 7 hours, or 8 hours elapses while the sample collector is left in place. In some embodiments, at most 5 minutes, 10 minutes, 15 minutes, 30 minutes, 45 minutes, 1 hour, 1.5 hours, 2 hours, 3 hours, 4 hours, 5 hours, 6 hours, 7 hours, or 8 hours elapses while the sample collector is left in place. In some embodiments, about 5 minutes, 10 minutes, 15 minutes, 30 minutes, 45 minutes, 1 hour, 1.5 hours, 2 hours, 3 hours, 4 hours, 5 hours, 6 hours, 7 hours, or 8 hours elapses while the sample collector is left in place

In some embodiments, the sample collector is placed within the displaceable shuttle. In some embodiments, the sample collector is placed within the first chamber. In some embodiments, the cap of the sample receptacle is closed. In some embodiments, closing the cap of the sample receptacle engages the displaceable shuttle. In some embodiments, placing a cap on the sample receptacle displaces the displaceable shuttle from the locking ring and results in piercing of the disruptable member.

In some embodiments, the sample is stabilized. In some embodiments, the sample collector continuously interacts with the stabilization buffer after the disruptable member is disrupted. In some embodiments, the sample collector is in continuous communication with the stabilization buffer for at least about 1, 2, 3, 4, 5, 6, 7, 8, 9, 10, 15, 20, 25, 30, 35, 40, 45, 50, 55, or 60 minutes. In some embodiments, the stabilization buffer is in continuous communication with the stabilization buffer for at least 1, 2, 3, 4, 5, 6, 7, 8, 9, 10, 11, 12, 14, 16, 18, 20, 22, 24, 30, 36, 48, 54, 60, 66, or 72 hours. In some embodiments, the sample collector is in continuous communication with the stabilization buffer for between 10 minutes and 72 hours.

In some embodiments, the preserved sample undergoes further analysis. In certain embodiments, the methods described herein comprise separating a biological material from the sample. In certain embodiments, separating a biological material from the sample comprises isolating the biological material from the sample using the methods or assays described herein. In certain embodiments, the biological material comprises nucleic acid, protein, a cell, or a combination thereof.

In some embodiments, the method or assay comprises isolation of the nucleic acid, protein, or a combination thereof from the cervicovaginal sample described herein. In some embodiments, the method or assay comprises isolation of the nucleic acid, protein, or a combination thereof from the sample described herein. In some embodiments, the method or assay comprises lysis of the cells in the sample. In some embodiments, the lysis is a chemical lysis, mechanical lysis, or a combination thereof. In some embodiments, chemical lysis comprises the addition of a lytic enzyme, a chaotropic agent, a detergent, or a combination thereof to the sample. In some embodiments, mechanical lysis comprises homogenizing, ultrasonicating, shearing, or shocking the cells. In some embodiments, the shocking comprises osmotic shock. In some embodiments, lysis results in release of the nucleic acids and proteins of the cell. In some embodiments, the method or assay comprises purification of the nucleic acids, proteins, or a combination thereof in the sample.

In some embodiments, the method or assay comprises extraction of the nucleic acids, proteins, or a combination thereof from the sample. In some embodiments, the nucleic acids is DNA, RNA, or combination thereof. In some embodiments, the RNA comprises mRNA, tRNA, rRNA, miRNA, siRNA, or a combination thereof. Extraction comprises organic phase extraction. In some embodiments, the method or assay comprises purification of the extracted nucleic acids, extracted proteins, or a combination thereof.

In some embodiments, the method or assay comprises sequencing the nucleic acid from the sample or the enriched sample. In some embodiments, the sequencing is whole-genome sequencing or whole-exome sequencing. In some embodiments, the sequencing is high-throughput sequencing. In some embodiments, the nucleic acid is sequences to a depth of at least 5×, 10×, 20×, 30×, 40×, 50×, 60×, 70×, 80×, 90×, 100×, 150×, 200×, 250×, 300×, or more than 300× coverage. In some embodiments, the sequencing is targeted sequencing, wherein one or more pre-selected nucleic acid targets are sequenced. In some embodiments, the one or more pre-selected nucleic acid targets is one or more biomarkers specific to endometriosis. In some embodiments, the sequencing comprises sequencing of 16S rRNA or 16S rDNA. In some embodiments, the method or assay comprises bisulfite treatment prior to the sequencing. In some embodiments, the methods or assays described herein comprise determining a methylation status of a nucleic acid in a nucleic acid sequence (i.e., methylated or not methylated). In some embodiments, the nucleic acid is a cytosine. In some embodiments, the methods or assays described herein comprise determining a methylation pattern of a nucleic acid sequence.

In some embodiments, the method or assay comprises determining, from a biological sample of the individual, an expression level of one or more microRNAs (miRs). In some embodiments, the method or assay comprises determining a methylation profile of one or more CpG sites. In some embodiments, the menstrualome footprint comprises a determining a measure of bacterial diversity in the biological sample. In some embodiments, the measure of bacterial diversity is an amount of at least 1, 2, 3, 4, 5, 6, 7, 8, 9, 10, or more bacteria.

### III. Kit

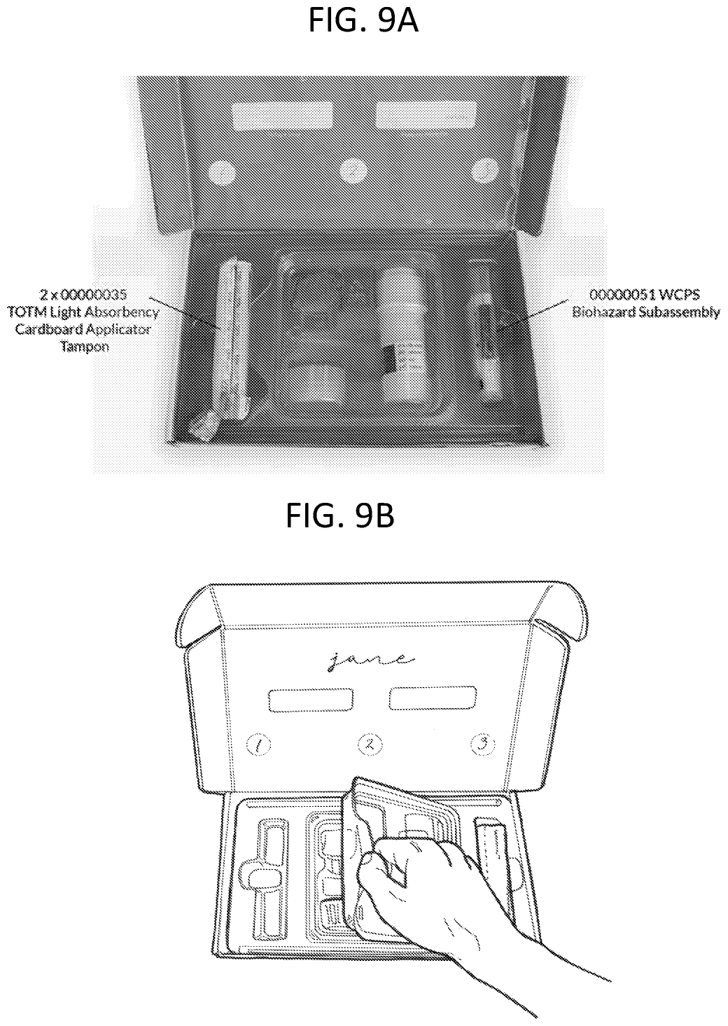

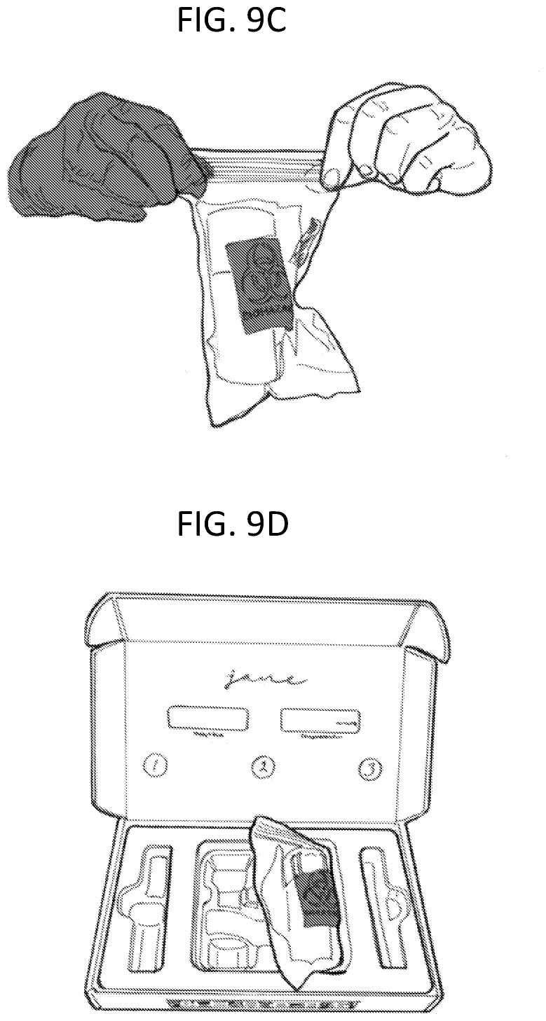

In some embodiments, disclosed herein is a kit comprising the sample collector disclosed herein, the sample collector disclosed herein and instructions for the use of the kit. In some embodiments, the kit comprises a biohazard bag. In some embodiments, the kit comprises an absorbent material. In some embodiments, the kit comprises gloves. In some embodiments, the contents are sterile and free of debris. In some embodiments, the kit comprises a retainer or snap-fit lid, wherein the retainer or snap-fit lid seals the system from foreign materials. In some embodiments, the kit is depicted in FIGS. 9A-9D.

### IV. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “subject,” “individual,” and “patient” are used interchangeably. None of the terms are to be interpreted as requiring the supervision of a medical professional (e.g., a doctor, nurse, physician's assistant, orderly, or hospice worker). In some cases, the subject is any animal, including mammals (e.g., a human or non-human animal). In one embodiment of the methods and compositions provided herein, the mammal is a human.

The term “nucleic acid,” as used herein, generally refers to a polymeric form of nucleotides of any length, either ribonucleotides and/or deoxyribonucleotides. Thus, these terms include, but are not limited to, single-, double-, or multi-stranded DNA or RNA, genomic DNA, complementary DNA (cDNA), guide RNA (gRNA), messenger RNA (mRNA), cell-free DNA (cfDNA), cell-free RNA (cfRNA), micro RNA (miRNA), DNA-RNA hybrids, or a polymer comprising purine and pyrimidine bases or other natural, chemically or biochemically modified, non-natural, or derivatized nucleotide bases.

The term “stabilization buffer,” as used herein, refers to an aqueous buffer that maintains a substantial proportion of cells in the stabilization buffer as intact, inviable (or nonproliferating) and substantially not expanding or contracting in size (e.g., +/−25%). In some embodiments, stabilization buffers herein comprise a preservative.

The term “preservative,” as used herein, refers to an agent added to a composition, such as a composition comprising cells, in order to prevent breakdown of biological components of the composition (e.g., cells). In some embodiments, preservatives herein maintain a composition comprising cells, keeping the cells inviable and intact without refrigeration. In embodiments herein, a preservative maintains the size of cells in the composition (i.e., does not cause the cells to shrink or expand). In some embodiments, a preservative may comprise a chelating agent (e.g., EDTA), a polyol (e.g., glycerol), a zwitterion (e.g., glycerol phosphate or pentaerythritol), an osomprotectants/pH buffer (e.g., Ca+, Cl−, K+, tartaric acid), or combinations thereof.

The section headings used herein are for organizational purposes only and are not to be construed as limiting the subject matter described.

### V. Examples

The following examples are included for illustrative purposes only and are not intended to limit the scope of the invention.

### Example 1: Collection and Preservation of a Menstrual Sample

A subject opens this kit pictured in FIG. 9A. The kit contains two tampons, a sample receptacle as described herein, a lid, a glove, and a biohazard bag. The tray contains a molded and removeable retainer as depicted in FIG. 9A. The subject inserts the tampon for at least 10 minutes to collect a sample. The tampon has a string. The sample may comprise menstrual blood, cervical mucus, cervicovaginal fluid, spotting blood (i.e., from between periods), amniotic fluid, a mucus plug, or other vaginal discharge, or a combination thereof. The subject then inserts the tampon comprising the sample into the first chamber of the sample receptacle. The cap of the same receptacle is screwed on such that the displaceable membrane disrupts the membrane on the buffer cup such that the sample collector and the buffer are in fluid communication. The subject then places the sealed sample receptacle in the biohazard bag as depicted in FIG. 9C. The biohazard bag is placed in the kit and the kit can be sealed and mailed for processing as depicted in FIG. 9D.

The movement of vibrations of the sample receptacle during shipping allow the cellular materials in the sample to elute into the stabilization buffer. This preserves the cells without rupturing them. This may also result in better admixture than if the sample collector was compressed. In some embodiments, the mixture of the sample and the stabilization buffer during shipping allow for removing a step in processing once the sample and sample receptacle has been received.

At the lab, the cap is replaced by the harvester mechanism. The harvester mechanism threads to the open end of the sample receptacle and compresses the tampon into the shuttle. The stabilization buffer comprising the sample is poured through the harvester mechanism into another device for processing.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 2: Sealing a Sample Receptacle Containing a Tampon with a String

A subject opens the kit pictured in FIG. 9A. The kit contains two tampons, a sample receptacle as described herein, a cap, a glove, and a biohazard bag. The tray contains a molded and removeable retainer as depicted in FIG. 9A. The subject inserts the tampon for at least 10 minutes to collect a sample. The tampon has a string. The sample may comprise menstrual blood, cervical mucus, cervicovaginal fluid, spotting blood (i.e., from between periods), amniotic fluid, a mucus plug, or other vaginal discharge, or a combination thereof. The subject then inserts the tampon comprising the sample into the first chamber of the sample receptacle. The string of the tampon comprising the sample either fully or partially remains outside of the sample receptacle as depicted in FIG. 12A. The cap of the receptacle contains V-shaped notches. When the cap is placed on top of the sample receptacle, the tampon string is pushed down into one of the notches located in the piercer or displaceable shuttle as depicted in FIG. 12B. As the cap is screwed on, the tampon string becomes caught in a notch located on the cap. As the cap is further screwed onto the sample receptacle, the tampon string remains caught in both notches and becomes wrapped around either a shuttle or piercer within the sample receptacle as depicted in FIG. 12C-FIG. 12F. When the cap is fully engaged, the tampon string is fully contained within the sample receptacle and does not disrupt the seal between the cap and the sample receptacle as depicted in FIG. 12G.

The subject then places the sealed sample receptacle in the biohazard bag as depicted in FIG. 9C. The biohazard bag is placed in the kit and the kit can be sealed and mailed for processing as depicted in FIG. 9D.

1. A system for extracting and preserving components of a biological sample of a subject comprising:
(a) a sample collector that collects and retains the biological sample from a vaginal canal of the subject; and
(b) a sample receptacle; wherein the sample receptacle comprises
(i) a first chamber that is configured to hold the sample collector;
(ii) a second chamber that is configured to contain a stabilization buffer;
(iii) a disruptable member between the first chamber and the second chamber; and
(iv) a displaceable shuttle, wherein displacement of the displaceable shuttle from the first chamber towards the second chamber is configured to disrupt the disruptable member such that there is fluid communication between the first chamber and the second chamber.

2. The system of claim 1, wherein the sample collector comprises an absorbent-diffuse material that collects, retains, or releases the biological material.

3. The system of claim 2, wherein the absorbent-diffuse material comprises one or more of a plant fiber material, a disposable material, a flushable material, a biodegradable material, and an organic material.

4. The system of any one of claims 1 to 3, wherein the sample collector is insertable in the vaginal canal.

5. The system of any one of claims 1 to 4, wherein the sample collector is a tampon, a pad, a plug, or a swab.

6. The system of any one of claims 1 to 5, wherein the sample collector continuously interacts with the stabilization buffer after the disruptable member is disrupted.

7. The system of claim 6, wherein the sample collector is in continuous communication with the stabilization buffer for about 10 minutes to about 48 hours.

8. The system of any one of claims 1 to 7, wherein the sample collector is not compressed.

9. The system of any one of claims 1 to 8, wherein the displaceable shuttle is configured such that it does not compress the sample collector.

10. The system of any one of claims 1 to 9, wherein the biological sample is released from the sample collector into the stabilization buffer.

11. The system of any one of claims 1 to 10, further comprising a locking ring comprising a first detent and a second detent, wherein the locking ring is located within the first chamber.

12. The system of claim 11, wherein the displaceable shuttle comprises a first end and a second end; wherein the first end of the displaceable shuttle comprises ridges;
wherein the displaceable shuttle is retained in the first chamber by the ridges fitting into the first detent on the locking ring; wherein the second end of the displaceable shuttle comprises a piercing end configured to disrupt the disruptable member.

13. The system of claim 11 or 12, wherein the displaceable shuttle is configured to hold the sample collector within the first chamber.

14. The system of any one of claims 11 to 13, wherein placing a cap on the receptacle displaces the displaceable shuttle from the first detent on the locking ring to the second detent on the locking ring.

15. The system of claim 14, wherein the cap is configured to engage the shuttle before fully closing.

16. The system of claim 14 or 15, wherein the cap comprises a material that is softer than the receptacle.

17. The system of any one of claims 14 to 15, wherein the ridges of the displaceable shuttle are configured to prevent the displaceable shuttle from piercing the disruptable member before the cap is placed

18. The system of any one of claims 1 to 17, wherein the second chamber comprises a buffer cup configured to contain the stabilization buffer.

19. The system of claim 18, wherein the buffer cup comprises an outer ring and an inner ring, wherein the outer ring is larger in diameter than the inner ring.

20. The system of claim 18 or 19, wherein the buffer cup is configured to contain about milliliters to 30 milliliters of the stabilization buffer.

21. The system of any one of claims 18 to 20, wherein the disruptable member is attached to the buffer cup.

22. The system of any one of claims 18 to 21, wherein the disruptable member comprises a foil lid or a plastic film.

23. The system of any one of claims 18 to 22, wherein the disruptable member is attached to the buffer cup by a method selected from the group consisting of heat sealing, adhesion, curing, and welding.

24. The system of any one of claims 1 to 23, wherein the sample receptacle comprises a compression mechanism configured to compress the sample collector.

25. The system of any one of claims 1 to 23, wherein the sample receptacle does not comprise a compression mechanism configured to compress the sample collector.

26. The system of any one of claims 1 to 25, further comprising a harvester mechanism.

27. The system of claim 26, wherein the harvester mechanism comprises a cap and a stem, wherein the stem is configured to be inserted into the sample receptacle and to compress the sample collector.

28. The system of claim 26 or 27, wherein the harvester mechanism comprises snap arms that interface with the sample collector.

29. The system of claim 28, wherein the snap arms of the harvester are configured to engage a lip on the sample receptacle to retain the sample collector within the displaceable shuttle.

30. The system of any one of claims 27 to 29, wherein the harvester is pressed onto the sample receptacle using an arbor press.

31. The system of any one of claims 26 to 29, wherein the harvester mechanism comprises a fluid passageway configured to allow the stabilization buffer and the biological sample to be moved from the second chamber of the sample receptacle into a second receptacle.

32. The system of claim 31, wherein the harvester comprises an air passageway that is separate from the fluid passageway.

33. The system of claim 31, wherein the fluid passageway is selected from the group consisting of a spout, a luer lock, a syringe port, and a one-way valve.

34. The system of any one of claims 1 to 33, wherein the stabilization buffer comprises a preservative.

35. The system of any one of claims 1 to 34, wherein the stabilization buffer is formulated to preserve one or more of intact cells, nucleic acids, DNA, RNA, protein or metabolite(s).

36. The system of any one of claims 1 to 35 wherein the system is configured to preserve one or more of more of intact cells, nucleic acids, DNA, RNA, protein or metabolite(s) for at least 72 hours at room temperature.

37. The system of any one of claims 1 to 36, wherein upon disruption of the disruptable member, the system is configured to retain the stabilization buffer between the first chamber and the second chamber when the device is rotated, placed horizontally, and/or inverted from its vertical position.

38. The system of claim 37, wherein upon rotation, horizontal placement, or inversion, the device is configured such that the stabilization buffer washes over the sample collector thereby releasing the biological sample from the sample collector into the stabilization buffer.

39. A kit comprising:
(a) the system of any one of claims 1 to 38; and
(b) instructions for the use of the kit.

40. The kit of claim 39, further comprising a biohazard bag and an absorbent material.

41. The kit of claim 39 or 40, further comprising gloves.

42. The kit of any one of claims 39 to 41, wherein the contents are sterile and free of debris.

43. The kit of any one of claims 39 to 42, wherein the kit comprises a retainer or snap-fit lid, wherein the retainer or snap-fit lid seals the system from foreign materials.

44. The system of claim 1, wherein the displaceable shuttle comprises a first notch or a first plurality of notches.

45. The system of claim 1, wherein the cap of the sample receptacle comprises a second notch or second plurality of notches.

46. The system of claim 44, wherein the first notch or plurality of notches is configured to catch a string appended to one end of the sample collector when the cap is placed on sample receptacle.

47. The system of claim 45, wherein the second notch or plurality of notches is configured to hold a string appended to one end of the sample collector when the cap is screwed on to the sample receptacle.

48. The system of claim 46 or 47, wherein after placing a cap on the sample receptacle, the string appended to one end of the sample collector remains entirely within the sample receptacle.

49. A sample receptacle, comprising:
(a) a tampon, wherein the tampon comprises a string;
(b) a displaceable shuttle, wherein the displaceable shuttle comprises a first notch or a first plurality of notches, wherein the first notch or first plurality of notches is configured to catch the tampon string; and
(c) a cap, wherein the cap comprises a second notch or a second plurality of notches, wherein said second notch or second plurality of notches is configured to hold the tampon string when the cap is screwed on to the sample receptacle.

50. The sample receptacle of claim 49, wherein the tampon string is fully contained within the sample receptacle after the cap is engaged with the sample receptacle.

51. The sample receptacle of claim 49, wherein the first notch or first plurality of notches is V-shaped.

52. The sample receptacle of claim 49, wherein the second notch or second plurality of notches is V-shaped.

53. A system for extracting and preserving components of a biological sample of a subject comprising:
(a) a sample collector that collects and retains the biological sample from a vaginal canal of the subject; and
(b) a sample receptacle; wherein the sample receptacle comprises
(i) a first chamber that is configured to hold the sample collector;
(ii) a second chamber that is configured to contain a stabilization buffer;
(iii) a disruptable member between the first chamber and the second chamber; and
(iv) a piercer, wherein the piercer is configured to pierce the disruptable member upon contact with said second chamber such that there is fluid communication between the first chamber and the second chamber.

54. The system of claim 53, wherein the sample collector comprises an absorbent-diffuse material that collects, retains, or releases the biological material.

55. The system of claim 54, wherein the absorbent-diffuse material comprises one or more of a plant fiber material, a disposable material, a flushable material, a biodegradable material, and an organic material.

56. The system of any one of claims 53 to 55, wherein the sample collector is insertable in the vaginal canal.

57. The system of any one of claims 53 to 56, wherein the sample collector is a tampon, a pad, a plug, or a swab.

58. The system of any one of claims 53 to 57, wherein the sample collector continuously interacts with the stabilization buffer after the disruptable member is disrupted.

59. The system of claim 58, wherein the sample collector is in continuous communication with the stabilization buffer for about 10 minutes to about 48 hours.

60. The system of any one of claims 53 to 59, wherein the sample collector is not compressed.

61. The system of any one of claims 53 to 60, wherein the piercer is configured such that it does not compress the sample collector.

62. The system of any one of claims 53 to 61, wherein the biological sample is released from the sample collector into the stabilization buffer.

63. The system of claim 53, wherein the piercer comprises a piercing end configured to disrupt the disruptable member.

64. The system of claim 53, wherein the piercer is configured to hold the sample collector within the first chamber.

65. The system of claim 53, wherein the second chamber is located within a cap of said sample receptacle.

66. The system of claim 65, wherein the cap comprises a buffer cup configured to contain the stabilization buffer.

67. The system of claim 66, wherein the buffer cup comprises an outer ring and an inner ring, wherein the outer ring is larger in diameter than the inner ring.

68. The system of claim 66 or 67, wherein the buffer cup is configured to contain about milliliters to 30 milliliters of the stabilization buffer.

69. The system of any one of claims 66 to 68, wherein the disruptable member is attached to the buffer cup.

70. The system of any one of claims 66 to 69, wherein the disruptable member comprises a foil lid or a plastic film.

71. The system of any one of claims 66 to 70, wherein the disruptable member is attached to the buffer cup by a method selected from the group consisting of heat sealing, adhesion, curing, and welding.

72. The system of any one of claims 53 to 71, wherein the sample receptacle comprises a compression mechanism configured to compress the sample collector.

73. The system of any one of claims 53 to 71, wherein the sample receptacle does not comprise a compression mechanism configured to compress the sample collector.

74. The system of any one of claims 53 to 73, further comprising a harvester mechanism.

75. The system of claim 74, wherein the harvester mechanism comprises a cap and a stem, wherein the stem is configured to be inserted into the sample receptacle and to compress the sample collector.

76. The system of claim 74 or 75, wherein the harvester mechanism comprises snap arms that interface with the sample collector.

77. The system of claim 76, wherein the teeth of the harvester are configured to engage a lip on the sample receptacle to retain the sample collector within the piercer.

78. The system of any one of claims 75 to 77, wherein the harvester is pressed onto the sample receptacle using an arbor press.

79. The system of any one of claims 75 to 78, wherein the harvester mechanism comprises a fluid passageway configured to allow the stabilization buffer and the biological sample to be moved from the second chamber of the sample receptacle into a second receptacle.

80. The system of claim 79, wherein the harvester comprises an air passageway that is separate from the fluid passageway.

81. The system of claim 79, wherein the fluid passageway is selected from the group consisting of a spout, a luer lock, a syringe port, and a one-way valve.

82. The system of any one of claims 53 to 81, wherein the stabilization buffer comprises a preservative.

83. The system of any one of claims 53 to 82, wherein the stabilization buffer is formulated to preserve one or more of intact cells, nucleic acids, DNA, RNA, protein or metabolite(s).

84. The system of any one of claims 53 to 83 wherein the system is configured to preserve one or more of more of intact cells, nucleic acids, DNA, RNA, protein or metabolite(s) for at least 72 hours at room temperature.

85. The system of any one of claims 53 to 84, wherein upon disruption of the disruptable member, the system is configured to retain the stabilization buffer between the first chamber and the second chamber when the device is rotated, placed horizontally, and/or inverted from its vertical position.

86. The system of claim 85, wherein upon rotation, horizontal placement, or inversion, the device is configured such that the stabilization buffer washes over the sample collector thereby releasing the biological sample from the sample collector into the stabilization buffer.

87. A kit comprising:
(a) the system of any one of claims 53 to 86; and
(b) instructions for the use of the kit.

88. The kit of claim 87, further comprising a biohazard bag and an absorbent material.

89. The kit of claim 87 or 88, further comprising gloves.

90. The kit of any one of claims 87 to 89, wherein the contents are sterile and free of debris.

91. The kit of any one of claims 87 to 90, wherein the kit comprises a retainer or snap-fit lid, wherein the retainer or snap-fit lid seals the system from foreign materials.

92. The system of claim 53, wherein the piercer comprises a first notch or a first plurality of notches.

93. The system of claim 53, wherein the cap of the sample receptacle comprises a second notch or second plurality of notches.

94. The system of claim 92, wherein the first notch or plurality of notches is configured to catch a string appended to one end of the sample collector when the cap is placed on sample receptacle.

95. The system of claim 93, wherein the second notch or plurality of notches is configured to hold a string appended to one end of the sample collector when the cap is screwed on to the sample receptacle.

96. The system of claim 94 or 95, wherein after placing a cap on the sample receptacle, the string appended to one end of the sample collector remains entirely within the sample receptacle.

97. A sample receptacle, comprising:
(a) a tampon, wherein the tampon comprises a string;
(b) a piercer, wherein the piercer comprises a first notch or a first plurality of notches, wherein said first notch or first plurality of notches is configured to catch the tampon string; and
(c) a cap, wherein the cap comprises a second notch or a second plurality of notches, wherein said second notch or second plurality of notches is configured to hold the tampon string when the cap is screwed on to the sample receptacle.

98. The sample receptacle of claim 97, wherein the tampon string is fully contained within the sample receptacle after the cap is engaged with the sample receptacle.

99. The sample receptacle of claim 97, wherein the first notch or first plurality of notches is V-shaped.

100. The sample receptacle of claim 97, wherein the second notch or second plurality of notches is V-shaped.
